(19) United States
(12) Patent Application Publication
(10) Pub. No.: US 2026/0216215 A1
(43) Pub. Date: Jul. 30, 2026

(54) COMPOSITIONS AND METHODS FOR TREATING OR PREVENTING PAIN OR OTHER DISORDERS

(71) Applicant: AIMA INC., Vancouver (CA)

(72) Inventor: Malihesadat POORMASJEDIMEIBOD, North Vancouver (CA)

(21) Appl. No.: 19/139,833

(22) PCT Filed: Dec. 15, 2023
(86) PCT No.: PCT/CA2023/051671

§ 371 Date: Jun. 16, 2025

Related U.S. Application Data
(60) Provisional application No. 63/433,377, filed on Dec. 16, 2022.

(51) Int. Cl. A61K 31/00; A61K 31/164; A61P 25/06; A61P 29/00

(52) U.S. Cl. CPC A61K 31/658; A61K 31/164; A61P 25/06; A61P 29/00

(57) ABSTRACT

In some example embodiments the invention relates to compositions comprising at least one N-acylethanolamine (NAE) and at least one of a cannabinoid, terpene, antioxidant or herbal extract. In other example embodiments the invention relates to methods for treating or preventing pain or pain-related symptoms in a patient, comprising administering to the patient a N-acylethanolamine (NAE) in combination with at least one of a cannabinoid, terpene, antioxidant or herbal extract, wherein the at least one of a cannabinoid, terpene, antioxidant or herbal extract is administered separately, sequentially or simultaneously to the at least one N-acylethanolamine. In other example embodiments the invention relates to treatment or prevention of other disorders such as inflammatory or psychological disorders or muscle spasm and spasticity, or to the improvement sleep duration or sleep quality, by administering at least one N-acylethanolamine (NAE) and at least one of a cannabinoid, terpene, antioxidant and herbal extract.

### CROSS-REFERENCE TO RELATED APPLICATIONS

This application claims priority from U.S. Application No. 63/433,377 filed 16 Dec. 2022 and entitled COMPOSITIONS AND METHODS FOR TREATING OR PREVENTING PAIN OR OTHER DISORDERS which is hereby incorporated herein by reference for all purposes. For purposes of the United States of America, this application claims the benefit under 35 U.S.C. § 119 of U.S. application No. 63/433,377 filed 16 Dec. 2022 and entitled COMPOSITIONS AND METHODS FOR TREATING OR PREVENTING PAIN OR OTHER DISORDERS.

### TECHNICAL FIELD

This application relates to compositions comprising at least one N-acylethanolamine (NAE) and at least one of a cannabinoid, terpene, herbal extract or antioxidant. In some embodiments the compositions and combinations of the compounds administered separately, sequentially or simultaneously can be used in methods for treatment or prevention of pain. In other embodiments the compositions and compounds can be used in methods for treatment or prevention of other disorders, such as inflammatory disorders, psychological disorders or spasticity. In other embodiments the compositions and compounds can be used in methods for improving sleep duration or sleep quality.

### BACKGROUND

All publications referred to herein are hereby incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

### Chronic Pain

Pain, recognized in some circumstances as a disease, is one of the most frequent reasons for visits to physicians, is among the most common reasons for taking medications, and is also a major cause of work disability. Severe chronic pain affects physical and mental functioning, quality of life and productivity. Further, chronic pain often imposes a significant financial burden on affected individuals, as well as on their families, their employers, their friends, their communities and the nation as a whole. The International Association for the Study of Pain defines pain as “an unpleasant sensory and emotional experience associated with actual or potential tissue damage or described in terms of such damage”.¹ This definition recognizes pain as a subjective experience with both psychological and sensory components. It also recognizes that tissue damage does not need to be present for pain to be experienced. Pain can be categorized along a variety of dimensions, including one of the most important divisions, nociceptive versus neuropathic pain. Nociceptive pain results from activity in neural pathways secondary to actual tissue damage or potentially tissue-damaging stimuli. Neuropathic pain is chronic pain that is initiated by nervous system lesions or dysfunction and can be maintained by a number of different mechanisms.²

Chronic pain refers to pain that lasts for more than three months, and the frequency of pain is at least once a week, accompanied by unpleasant feelings and emotions, and may be accompanied by existing or potential tissue damage.³ According to the International Classification of Diseases (ICD) established by the World Health Organization (WHO), chronic pain is divided into the following categories: chronic primary pain, chronic cancer pain, chronic postoperative pain and post-traumatic pain, chronic neuropathic pain, chronic head and jaw facial pain, chronic visceral pain, chronic skeletal muscle pain, etc.⁴

Chronic pain is a common type of pain, is the most common clinical disease, and one of the diseases that seriously affects human health. Epidemiological studies have shown that the proportion of people suffering from chronic pain is as high as about 20% to 30% of the population.^{5,6} Studies have shown that some chronic pain develops from acute trauma, such as chronic postoperative pain, phantom limb pain, and neuropathic pain.

### Pelvic Pain and Dysmenorrhea

It is estimated that pelvic pain affects up to 25% of the female population. 7,8,9 Although there are many causes of pelvic pain, dysmenorrhea is a common symptom, which may exist alone or related to other pelvic pain symptoms, additional pelvic pain and other symptoms. Dysmenorrhea is a medical term used to describe pain experienced during menstruation. This pain is thought to originate in the uterus and is usually felt in the pelvis or lower abdomen, but may be felt in the lower back or thighs. Although dysmenorrhea may be the only symptom, it may also be related to other pain-related symptoms, such as bladder pain (usually related to frequent urination, urgency or nocturia), bowel pain (usually related to bloating, diarrhea or constipation), musculoskeletal pain (usually felt as pain, sharp pain, tingling or sudden pain), pain associated with intercourse, or other systemic symptoms including fatigue, nausea, poor sleep, headache, anxiety, or depression.¹⁰ Dysmenorrhea is a major problem affecting a large proportion of women, affecting 16%-91% of women of reproductive age, with severe pain in 2%-29% of the women studied.¹¹ For example, a study of 16 to 18-year-old women in Australia showed that although 93% of women experience some pain during menstruation, 21% of women experience severe pain, which is usually related to interrupted life activities and school absenteeism¹². This is a common reason for visits and hospitalizations in the emergency department.

There are two types of dysmenorrhea-primary dysmenorrhea and secondary. In dysmenorrhea, recurrent and painful menstrual cramps occur one or two days before and after menstrual bleeding, and is felt in the lower abdomen, back, or thigh and is mild to severe in nature, typically lasting 12 to 72 hours. Nausea, vomiting, fatigue, and even diarrhea may also be seen in this type of condition. Primary dysmenorrhea affects approximately 45% to 95% of menstruating women. Secondary dysmenorrhea is characterized by pain due to a disorder or infection in the woman's reproductive organs; the pain usually begins earlier in the menstrual cycle and lasts longer than common menstrual cramps.^{13,14}

### Chronic Pain Management

While numerous non-pharmacological techniques are used to treat chronic pain, including transcutaneous electrical nerve stimulation (TENS), acupuncture, physical therapy, and psychotherapy, pharmacotherapy may be the most widely used method for treating pain, albeit with variable success. Commonly used clinical drugs include the following categories: opioids, non-steroidal antipyretic analgesics, antiepileptic drugs, antidepressants, etc.^{15,16} However, these drugs commonly have negative characteristics such as addiction, tolerance, or adverse effects on the gastrointestinal system, vascular system, blood coagulation system, liver and kidney, or are ineffective in some patients.¹⁷

Various classes of drugs are used to treat dysmenorrhea such as non-steroidal anti-inflammatory drugs (NSAIDs), glyceryl trinitrate, progestin regimens and levonorgestrel intrauterine system (LN-IUS).^{18,19} However, these types of treatments may produce adverse reactions such as nausea, dyspepsia, peptic ulcers, and diarrhea, stomach pain, heart attacks, strokes, constipation, gas, heartburn, vomiting, and dizziness, which make them unsuitable for regular use or poor management of pain. Hormonal management including (but not limited to) oral contraceptives, oral progestins, or gonadotropin releasing hormone agonists may improve symptoms in some women, especially in the short term. However, these managements may not be effective enough for the management of pain and pain-related symptoms, may cause adverse reactions, or may not be accepted by some women.^{20,21}

There is a continuing need to develop new treatments for pain management, especially in view of the ongoing opioid epidemic that has resulted in prescription opioid analgesics leading to dependence in many patients, and abuse by other elements of society.

The foregoing examples of the related art and limitations related thereto are intended to be illustrative and not exclusive. Other limitations of the related art will become apparent to those of skill in the art upon a reading of the specification.

### SUMMARY

One aspect of the invention relates to a composition comprising at least one N-acylethanolamine (NAE) and at least one of a cannabinoid, terpene, herbal extract or antioxidant. In some embodiments the at least one N-acylethanolamine (NAE) is a ligand of a cannabinoid receptor. In some embodiments the at least one N-acylethanolamine (NAE) comprises N-palmitoylethanolamide (PEA). In some embodiments the at least one N-acylethanolamine (NAE) comprises Anandamide. In some embodiments the at least one cannabinoid comprises two cannabinoids, the cannabinoids being cannabidiol (CBD) and cannabigerol (CBG). In some embodiments the at least one cannabinoid is CBDA. In some embodiments the at least one cannabinoid is CBGA. In some embodiments, the at least one antioxidant is resveratrol.

In another aspect, the invention relates to methods for treating or preventing pain and/or pain-related symptoms in a patient, comprising administering to the patient at least one N-acylethanolamine (NAE) in combination with at least one of a cannabinoid, terpene, herbal extract or antioxidant, wherein the at least one of a cannabinoid, terpene, herbal extract or antioxidant is administered separately, sequentially or simultaneously to the at least one N-acylethanolamine (NAE). In some embodiments the active pharmaceutical ingredients may be deployed in various dosage forms, drug delivery systems or methods of use.

In other aspect, the invention relates to methods for treating or preventing an inflammatory disorder or a psychological disorder in a patient, comprising administering to the patient at least one N-acylethanolamine (NAE) in combination with at least one of a cannabinoid, terpene herbal extract, or antioxidant, wherein the at least one of a cannabinoid, terpene, herbal extract or antioxidant is administered separately, sequentially or simultaneously to the at least one N-acylethanolamine (NAE).

In other aspects, the invention relates to use of compositions comprising at least one N-acylethanolamine (NAE) and at least one of a cannabinoid, terpene, herbal extract or antioxidant for treatment or prevention of pain and/or pain-related symptoms in a patient, or for treatment or prevention of an inflammatory disorder or a psychological disorder in a patient.

In another aspect of the invention, the invention relates to use of compositions comprising at least one N-acylethanolamine (NAE) and at least one of a cannabinoid, terpene, herbal extract or antioxidant for treatment or prevention of spasticity, muscle cramping or spasticity pain-related symptoms in a patient.

In another aspect of the invention, the invention relates to use of compositions comprising at least one N-acylethanolamine (NAE) and at least one of a cannabinoid, terpene, herbal extract or antioxidant for improving sleep duration or sleep quality in a subject.

In another aspect of the invention, the invention relates to use of compositions comprising at least one N-acylethanolamine (NAE) and at least one of a cannabinoid, terpene, herbal extract or antioxidant to inhibit Cox-1 or Cox-2 enzymes and/or to inhibit prostaglandin production.

In addition to the exemplary aspects and embodiments described above, further aspects and embodiments will become apparent by study of the following detailed descriptions.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description is to be regarded in an illustrative, rather than a restrictive, sense.

In some embodiments the compositions of the invention may comprise at least one N-acylethanolamine (NAE) and at least one of a cannabinoid, terpene, herbal extract or antioxidant. In some embodiments the compositions may comprise a combination of one or more of a cannabinoid, terpene, herbal extract or antioxidant. By way of examples, the compositions may comprise at least one N-acylethanolamine (NAE) in combination with (1) at least one cannabinoid; or (2) at least one terpene; or (3) at least herbal extract; or (4) at least antioxidant; or (5) combinations of at least one cannabinoid, terpene, herbal extract or antioxidant. By way of further examples the compositions may comprise at least one N-acylethanolamine (NAE) in combination with multiple cannabinoids; or at least one N-acylethanolamine (NAE) in combination with at least one cannabinoid and at least one terpene; or at least one N-acylethanolamine (NAE) in combination with at least one herbal extract; or at least one N-acylethanolamine (NAE) in combination with multiple herbal extracts; or at least one N-acylethanolamine (NAE) in combination with multiple antioxidants. Many other example combinations and permutations comprising at least one N-acylethanolamine (NAE) and at least one of a cannabinoid, terpene herbal extract or antioxidant are within the scope of the invention. In some embodiments two or more of the compounds of the invention, namely the N-acylethanolamine (NAE), cannabinoid, terpene, herbal extract or antioxidant, may exhibit a synergistic effect when administered in combination.

In some embodiments the compositions and combinations of the compounds can be used for treatment and/or prevention of pain or pain-related symptoms. In some embodiments methods of treating and/or preventing pain or pain-related symptoms are disclosed comprising administering to a patient at least one N-acylethanolamine (NAE) in combination with at least one cannabinoid, terpene, herbal extract or antioxidant, wherein the at least one cannabinoid, terpene, herbal extract or antioxidant is administered separately, sequentially or simultaneously to the at least one N-acylethanolamine (NAE). In some embodiments the compositions and combinations of the compounds can be used for treatment and/or prevention of inflammatory disorders, psychological disorders or spasticity, which in some cases are associated with chronic pain. In some embodiments the compositions and combinations of the compounds can be used for improving sleep duration or sleep quality. In some embodiments the compositions and combinations of the compounds can be used to inhibit Cox-1 or Cox-2 enzymes and/or to inhibit prostaglandin production. Other therapies and combination therapies are described herein.

### N-Acylethanolamines

N-acylethanolamines (NAEs) are a family of endogenous biologically active fatty acid amides comprising an acyl group linked to ethanolamine. NAEs are involved in lipid signaling and are involved in the modulation of different physiological processes including pain, stress, anxiety, appetite, cardiovascular function, inflammation, neurotransmission, and fertility. Names for specific NAEs may be encountered with either the suffix “amide” or “amine”, though these suffixes each refer to the single nitrogen atom of ethanolamine that links the compound together. It is termed “amine” in ethanolamine when considering the nitrogen atom as a free terminal nitrogen in that subunit, while it is termed “amide” when the nitrogen is considered in association with the adjacent carbonyl group of the acyl subunit.

Examples of N-acylethanolamines include:
1. Anandamide (N-arachidonoylethanolamine; NAE) or arachidonoylethanolamine (AEA:C22H37NO2; 20:4, ω-6) is the ligand of both cannabinoid receptors and the vanilloid receptor that attenuates pain sensation.
2. N-Palmitoylethanolamide (PEA:C18H37NO2; 16:0), also referred to as N-(2-hydroxyethyl)-hexadecanamide, has been assigned CAS number 544-31. It is a ligand at CB2 receptors and has anti-inflammatory activity and attenuates pain sensation in mammals.
3. N-Oleoylethanolamine (OEA:C20H39NO2; 18:1, ω-9), the plasma levels of which are positively correlated with positive mood and emotions.
4. N-Docosahexaenoylethanolamine (DHEA:C24H37NO2; 22:6, ω-3), or Anandamide (22:6, n-3) “synaptamide”, acts at CB2 receptors.
5. N-Docosatetraenoylethanolamine (DEA:C24H41NO2; 22:4, ω-6) act on both CB1 and CB2 receptors.
6. N-Eicosapentaenoylethanolamide (EPEA:C22H35NO2; 20:5, ω-3) or Anandamide (20:5, n-3) act on CB2 receptors in combination with PPAR-gamma.
In some embodiments the compositions and methods of the invention comprise N-Palmitoylethanolamide (PEA) or analogs or derivatives thereof. Palmitoylethanolamide (PEA) is interchangeably referred to as Palmitoylethanolamine in some instances, depending on author. PEA is one of the most frequently occurring and studied NAEs. Preclinical and clinical studies suggest PEA may potentially be useful in a wide range of therapeutic areas, including eczema, pain and neurodegeneration and at the same time to be essentially devoid of unwanted effects in humans.^{57,58,59,60,61,62,63,64,65,66,67,68,69} PEA is currently marketed for veterinary use (skin conditions, Redonyl™, [Innovet]606165) and as a nutraceutical in humans (Normast™666261, Pelvilen™ [Epitech]676362), PeaPure™ [JP Russel Science Ltd]686463) in some European countries (e.g. Italy, Spain; it is sold as a food supplement in other countries, such as the Netherlands). It also is a constituent of a cream (Physiogel AI™, Stiefel696264) marketed for dry skin. In clinical trials^{57,58,59,60,62,63,66,67,68,69}, PEA was used for periods ranging from 14 days to 120 days, and the doses ranged from 300 mg to 1200 mg daily. The administration form of PEA was in most cases oral tablets except some occasional use of sublingual formulations (sachets), and the commonest form of evaluation was the Visual Analog Scale (VAS), where the patient makes a subjective assessment of her/his pain level on a 10 cm line where the left side represents no pain, and the right side represents the worst imaginable pain. With one exception (possibly a ‘floor effect’), all available clinical trials reported significantly reduced pain intensity and an almost complete absence of unwanted effects, the latter confirming early field studies of PEA in healthy individuals. Case reports, pilot studies and clinical trials investigating the effect of PEA for the treatment of pain have been published as well.^{62,69}

Emanuela Stochino Loi evaluated the effectiveness of the ultramicronized-palmitoylethanolamide (um-PEA) and co-micronised palmitoylethanolamide/polydatin m (PEA/PLD) in the management of chronic pelvic pain related to endometriosis.¹³⁵ Thirty symptomatic women with laparoscopy-confirmed endometriosis were treated with PEA twice daily for 10 days followed by m (PEA/PLD) twice daily for 80 days. Chronic pelvic pain, deep dyspareunia, dysmenorrhea, dyschezia, quality of life and psychological well-being were significantly improved in these patients.

As indicated above, in some embodiments the compositions and methods of the invention may comprise PEA or analogs and derivatives thereof. In some embodiments the human daily dose of PEA is between 1 mg and 5000 mg. Preferably the effective human daily dose of PEA is between 200 mg and 1000 mg. More preferably the effective human daily dose of PEA is between 250 mg and 750 mg.

In some embodiments compositions and methods of the invention may include one or more of the following types of N-acylethanolamines in addition to or in substitution for PEA: Anandamide, N-Oleoylethanolamine, N-Docosahexaenoylethanolamine, N-Docosatetraenoylethanolamine, N-Eicosapentaenoylethanolamide, or analogs and derivatives thereof.

### Cannabinoids

Cannabinoids are a group of chemicals known to activate cannabinoid receptors in cells. These chemicals are found in cannabis plants and are also produced endogenously in humans and other animals. Cannabinoids produced endogenously are termed endocannabinoids.³² Synthetic cannabinoids are chemicals with similar structures to plant cannabinoid or endocannabinoids. Cannabinoids possess the characteristics of being cyclic molecules exhibiting various beneficial properties such as the ability to easily cross the blood-brain barrier, weak toxicity and few side effects.

References to cannabinoids, particularly with regard to therapeutic use, will be understood to also encompass pharmaceutically acceptable salts of the cannabinoids. The term “pharmaceutically acceptable salts” refers to salts or esters prepared from pharmaceutically acceptable non-toxic bases or acids, including inorganic bases or acids and organic bases or acids, as would be well known to persons skilled in the art. Many suitable inorganic and organic bases are known in the art.

In some embodiments compositions and methods of the invention may include or use one or more of the following cannabinoids: Cannabigerolic Acid (CBGA), Cannabigerolic Acid monomethylether (CBGAM), Cannabigerol (CBG), Cannabigerol monomethylether (CBGM), Cannabigerovarinic Acid (CBGVA), Cannabigerovarin (CBGV), Cannabichromenic Acid (CBCA), Cannabichromene (CBC), Cannabichromevarinic Acid (CBCVA), Cannabichromevarin (CBCV), Cannabidiolic Acid (CBDA), Cannabidiol (CBD), Cannabidiol monomethylether (CBDM), Cannabidiol-C4 (CBD-C4), Cannabidivarinic Acid (CBDVA), Cannabidivarin (CBDV), Cannabidiorcol (CBD-C1), Tetrahydrocannabinolic acid A (THCA-A), Tetrahydrocannabinolic acid B (THCA-B), Tetrahydrocannabinolic Acid (THCA), Tetrahydrocannabinol (THC), Tetrahydrocannabinolic acid C4 (THCA-C4), Tetrahydrocannbinol C4 (THC-C4), Tetrahydrocannabivarinic acid (THCVA), Tetrahydrocannabivarin (THCV), Tetrahydrocannabiorcolic acid (THCA-C1), Tetrahydrocannabiorcol (THC-C1), Δ7-cis-iso-tetrahydrocannabivarin, Δ8-tetrahydrocannabinolic acid (Δ8-THCA), Cannabivarinodiolic (CBNDVA), Cannabivarinodiol (CBNDV), Δ8-tetrahydrocannabinal (Δ8-THC), Δ9-tetrahydrocannabinol (Δ9-THC), Cannabicyclolic acid (CBLA), Cannabicyclol (CBL), Cannabicyclovarin (CBLV), Cannabielsoic acid A (CBEA-A), Cannabielsoic acid B (CBEA-B), Cannabielsoin (CBE), Cannabivarinselsoin (CBEV), Cannabivarinselsoinic Acid (CBEVA), Cannabielsoic Acid (CBEA), Cannabielvarinsoin (CBLV), Cannabielvarinsoinic Acid (CBLVA), Cannabinolic acid (CBNA), Cannabinol (CBN), Cannabivarinic Acid (CBNVA), Cannabinol methylether (CBNM), Cannabinol-C4 (CBN-C4), Cannabivarin (CBV), Cannabino-C2 (CBN-C2), Cannabiorcol (CBN-C1), Cannabinodiol (CBND), Cannabinodiolic Acid (CBNDA), Cannabinodivarin (CBDV), Cannabitriol (CBT), 10-Ethoxy-9-hydroxy-Δ8a-tetrahydrocannabinol, 8,9-Dihydroxy-Δ8a(10a)-tetrahydrocannabinol (8,9-Di-OH-CBT-C5), Cannabitriolvarin (CBTV), Ethoxy-cannabitriolvarin (CBTVE), Dehydrocannabifuran (DCBF), Cannbifuran (CBF), Cannabichromanon (CBCN), Cannabicitran (CBT), 10-Oxo-Δ8a(10a)-tetrahydrocannabinol (OTHC), Δ9-cis-tetrahydrocannabinol (cis-THC), Cannabiripsol (CBR), 3,4,5,6-tetrahydro-7-hydroxy-alpha-alpha-2-trimethyl-9-n-propyl-2,6-methano-2H-1-benzoxocin-5-methanol (OH-iso-HHCV), Trihydroxy-delta-9-tetrahydrocannabinol (triOH-THC), Yangonin, Epigallocatechin gallate, Dodeca-2E,4E,8Z,10Z-tetraenoic acid isobutylamide, and Dodeca-2E,4E-dienoic acid isobutylamide. In some embodiments the one or more cannabinoids may be in the form of an extract from a cannabis plant or hemp plant. In some embodiments the cannabinoid may be in an essentially pure form. In some embodiments the cannabinoid may be in synthetic form. In some embodiments compositions and methods of the invention may comprise 1, 2, 3, 4 or more cannabinoids.

Preferably the cannabinoids are present in a dose effective to relieve pain. A low dose of cannabinoids is defined as an effective human daily dose of cannabinoids of below 10 mg and a high dose of cannabinoids is defined as an effective human daily dose of cannabinoids of 500 mg or above. An intermediate dose is defined as being between 10 mg and 500 mg. Preferably the effective human daily dose of cannabinoids is between 25 mg and 200 mg. More preferably the effective human daily dose of cannabinoids is between 50 mg and 150 mg.

The scope of this disclosure also extends to derivatives of cannabinoids that retain the desired activity, such as pain relief. Derivatives that retain substantially the same activity as the starting material, or more preferably exhibit improved activity, may be produced according to standard principles of medicinal chemistry, which are well known in the art. Such derivatives may exhibit a lesser degree of activity than the starting material, so long as they retain sufficient activity to be therapeutically effective. Derivatives may exhibit improvements in other properties that are desirable in pharmaceutically active agents such as, for example, improved solubility, reduced toxicity, enhanced uptake, etc.

### Endocannabinoid System (ECS)

The endocannabinoid system (ECS) is involved in regulating a variety of physiological and cognitive processes including fertility, pregnancy, during pre- and postnatal development, appetite, pain and pleasure sensation, immune system, mood, and memory, and in mediating the pharmacological effects of cannabis.^{32,33}

Endocannabinoid receptors in the brain interact with cannabinoids from different sources, including:
1. Endocannabinoids (brain derived, e.g., from foods (Omega-3s and Omega-6s)). Endocannabinoids are endogenous lipid-based retrograde neurotransmitters that bind to cannabinoid receptors, and cannabinoid receptor proteins that are expressed throughout the mammalian central nervous system (including the brain) and peripheral nervous system.³³
2. Phytocannabinoids (plant derived, e.g., from buds, tinctures, extracts, including tetrahydrocannabinol (THC), cannabidiol (CBD), cannabinol (CBN), etc.). Plant cannabinoids or phyto-cannabinoids can also be isolated such that they are “essentially pure” compounds. These isolated cannabinoids are essentially free of the other naturally occurring compounds, such as, other minor cannabinoids and molecules such as terpenes. At least 85 different cannabinoids isolated from cannabis exhibit varied effects. Phytocannabinoid Δ9-tetrahydrocannabinol (THC) is the primary psychoactive compound of cannabis. Cannabidiol (CBD) is another major constituent of the plant, and comprises up to 40% extracts of plant resin. Essentially pure compounds have a degree of purity up to at least 95% by total weight. Some essentially pure cannabinoids (whether synthetic or isolated) have been suggested to be neuroprotective agents, either by direct antagonism of the NMDA receptor or by reducing the influx of calcium ions into the cell by another means such as binding with cannabinoid receptors.³⁴
3. Synthetic cannabinoids (such as tetrahydrocannabinol (THC)).

Cannabis sativa has been used for medical, recreational, and spiritual purposes for thousands of years. The utilization of cannabis for pain can be traced back to ancient Chinese texts, dating to 2900 B.C.³⁵ The Shennong Ben Cao Jing, a Chinese encyclopedia on agriculture and medicine, contains the oldest written record of cannabis as a medicine, recommending cannabis for constipation, rheumatic pain, female reproductive tract disorders, and malaria.

Modern scientific studies respecting cannabinoids have provided increasing amounts of preclinical and clinical evidence about their beneficial pharmacological effects, including pain relief. Among hundreds of compounds identified in cannabis so far, two phytocannabinoids, D9-tetrahydrocannabinol (D9-THC) and cannabidiol (CBD), are considered the primary active components of C. sativa and have been shown to produce pain relief in animal models.^{36,37,38,39} A review of literature published on Medline between 1975 and 2017 identified five clinical studies that evaluated the effect of THC or CBD on controlling cancer pain. Five studies that evaluated THC oil capsules, THC:CBD oromucosal spray (nabiximols), or THC oromucosal sprays found some evidence of cancer pain reduction associated with these therapies. A variety of doses ranging from 2.7-43.2 mg/day THC and 0-40 mg/day CBD were administered. Higher doses of THC were correlated with increased pain relief in some studies. One study found that significant pain relief was achieved in doses as low as 2.7-10.8 mg THC in combination with 2.5-10.0 mg CBD. Some reported side effects include drowsiness, hypotension, mental clouding, and nausea and vomiting.⁴⁰

It has been shown previously that CBD administered as a purified compound can partially relieve neuropathic pain. This was shown using the neuropathic pain model of chronic constriction injury of the rat sciatic nerve and testing the effectiveness of the test article with thermal and mechanical hyperalgesia and mechanical allodynia. These animal models are used to predict the effectiveness of a test compound on neuropathic pain.^{41,42} Patent GB2439393 describes a plant extract comprising a defined ratio of CBD to THC that is more effective at treating peripheral neuropathic pain than the purified components alone. The ratio of CBD to THC which is effective is between 20:1 to 28:1.⁴³ Published patent application PCT/GB2006/004063 (WO2007052013A1) describes the use of an extract of cannabis wherein the THC to CBD ratio is about 1:1. The extract was found to be beneficial in the treatment of peripheral neuropathic pain that is characterised by post-herpetic neuralgia.⁴⁴

Sativex⁴⁵ is a buccal spray containing 27 mg/ml THC and 25 mg/ml CBD. GW Pharma and others have conducted numerous clinical studies to assess the safety and efficacy of THC/CBD preparations such as Sativex for the treatment of spasticity and pain^{46,47,48,49,50}.

In one randomised, double-blind, placebo-controlled clinical trial, Sativex was shown to successfully treat neuropathic pain characterised by allodynia. In this trial, 63 patients were randomised to receive Sativex and 62 received a placebo. The mean reduction in pain intensity scores (primary outcome measure) was greater in patients receiving Sativex than placebo (mean adjusted scores −1.48 points vs. −0.52 points on a 0-10 numerical rating scale (p=0.004; 95% CI: −1.59, −0.32). Improvements in Neuropathic Pain Scale composite score (p=0.007), sleep NRS (p=0.001), dynamic allodynia (p=0.042), punctate allodynia (p=0.021), Pain Disability Index (p=0.003) and Patient's Global Impression of Change (p<0.001) were similarly greater on Sativex vs. placebo).⁴⁸

### Terpenes

Terpenes, also referred to as terpenoids or isoprenoids, are natural volatile aromatic compounds found in several organisms belonging to the animal and plant kingdoms. Terpenes contain a carbon and hydrogen scaffold (terpenes) or a carbon, hydrogen, and oxygen scaffold (terpenoids). They constitute a large class of natural products with >55,000 known compounds structurally diversified. Typical structures contain carbon skeletons represented by “(C5) n” and are classified as hemiterpenes (C5), monoterpenes (C10), sesquiterpenes (C15), diterpenes (C20), sesterterpenes (C25), triterpenes (C30) and tetraterpenes (C40).^{51,52} Within the context of this disclosure, the term “terpene” includes the α- (alpha), β- (beta), γ- (gamma), oxo-, isomers, or any combinations thereof.

Terpenoids are pharmacologically versatile: they are lipophilic, interact with cell membranes, neuronal and muscle ion channels, neurotransmitter receptors, G-protein coupled (odorant) receptors, second messenger systems and enzymes. All the terpenoids discussed herein are Generally Recognized as Safe, as attested by the US FDA as food additives, or by the Food and Extract Manufacturers Association and other world regulatory bodies.

Terpenes and terpenoids have been extensively used in various industrial sectors as flavors, fragrances and spices and are also used in perfumery and cosmetic products, for example as skin preparation agents, as well as food additives.⁵³ Terpenoid components in concentrations above 0.05% are considered of pharmacological interest in the pharmaceutical industry. In addition to being used as excipients to enhance skin penetration, they are also useful as active principles of drugs. Growing interest in the clinical application of these compounds is assigned by the broad range of the biological properties of terpenoids that have been described, including cancer chemopreventive effects, antimicrobial, antifungal, antiviral, antihyperglycemic, analgesic, anti-inflammatory and antiparasitic activities.

Lee et al evaluated the efficacy of Rowatinex terpene mixture compared to ibuprofen in patients with chronic prostatitis/chronic pelvic pain syndrome.⁵⁴ Rowatinex is composed of pinene (31%, 24.8 mg α-pinene, 6.2 mg β-pinene), camphene (15%, 15.0 mg), anethol (4%, 4.0 mg), borneol (10%, 10.0 mg), cineol (3%, 3.0 mg) and fenchone (4%, 4.0 mg) in olive oil. Rowatinex 200 mg t.i.d. gave significant symptomatic relief with on par pain relief efficacy to ibuprofen 600 mg t.i.d.⁵⁴

The analgesic properties of monoterpenes and sesquiterpenes, including their patent status, pharmacological properties, indications, action mechanism and exemplary formulations have been described by Guimarães et al.⁵⁵ A summary of the pharmacological properties and preclinical studies of selected Cannabis terpenes has been published by Liktor-Busa et al.⁵⁶

Terpenes also play important roles in cannabinoid-comprising products, affecting the aroma of the product. Terpenes can correct or enhance the effect of the cannabinoids, so that in many cases, (much) less active components may be needed to attain the desired effects.

Terpenes are what gives each cannabis strain its unique smell and taste. It is generally known that different cannabis strains produce different biological or medicinal effects or results and this is in part due to different terpene profiles. For example, even if 2 strains have the same cannabinoid content (THC, CBD, etc.), they often have different medical benefits and overall effects.

In some embodiments the at least one terpene of the present disclosure comprises one or more of:
Alpha Bisabolol
Alpha Pinene
Beta Caryophyllene
Beta Pinene
Borneol
Camphene
Caryophyllene Oxide
Cineole
Delta 3 Carene
Eucalyptol
Fenchol
Fenchone
Geraniol
Guaiol
Humulene
Isopulegol
Limonene
Linalool
Myrcene
Nerol
Nerolidol
Ocimene
Phytol
Pulegone
Terpinene
Terpineol
Terpinolene and/or
Valencene

In some embodiments, the compositions and methods of the invention may include or use one or more of the following terpenes: 7,8-dihydro-alpha-ionone, 7,8-dihydro-beta-ionone, Acetanisole, Acetic Acid, Acetyl Cedrene, Anethole, Anisole, Benzaldehyde, Bergamotene (Alpha-cis-Bergamotene) (Alpha-trans-Bergamotene), Bisabolol (Beta-Bisabolol), Alpha, Bisabolol, Borneol, Bornyl Acetate, Butanoic/Butyric Acid, Cadinene (Alpha-Cadinene) (Gamma-Cadinene), Cafestol, Caffeic acid, Camphene, Camphor, Capsaicin, Carene (Delta-3-Carene), Carotene, Carvacrol, Dextro-Carvone, Laevo-Carvone, Caryophyllene (Beta-Caryophyllene), Caryophyllene oxide, Cedrene (Alpha-Cedrene) (Beta-Cedrene), Cedrene Epoxide (Alpha-Cedrene Epoxide), Cedrol, Cembrene, Chlorogenic Acid, Cinnamaldehyde, Alpha-amyl-Cinnamaldehyde, Alpha-hexyl-Cinnamaldehyde, Cinnamic Acid, Cinnamyl Alcohol, Citronellal, Citronellol, Cryptone, Curcumene (Alpha-Curcumene) (Gamma-Curcumene), Decanal, Dehydrovomifoliol, Diallyl Disulfide, Dihydroactinidiolide, Dimethyl Disulfide, Eicosane/Icosane, Elemene (Beta-Elemene), Estragole, Ethyl acetate, Ethyl Cinnamate, Ethyl maltol, Eucalyptol/1,8-Cineole, Eudesmol (Alpha-Eudesmol) (Beta-Eudesmol) (Gamma-Eudesmol), Eugenol, Euphol, Farnesene, Farnesol, Fenchol (Beta-Fenchol), Fenchone, Geraniol, Geranyl acetate, Germacrenes, Germacrene B, Guaia-1 (10), 11-diene, Guaiacol, Guaiene (Alpha-Guaiene), Gurjunene (Alpha-Gurjunene), Herniarin, Hexanaldehyde, Hexanoic Acid, Humulene (Alpha-Humulene) (Beta-Humulene), Ionol (3-oxo-alpha-ionol) (Beta-Ionol), Ionone (Alpha-Ionone) (Beta-Ionone), Ipsdienol, Isoamyl Acetate, Isoamyl Alcohol, Isoamyl Formate, Isoborneol, Isomyrcenol, Isopulegol, Isovaleric Acid, Isoprene, Kahweol, Lavandulol, Limonene, Gamma-Linolenic Acid, Linalool, Longifolene, Alpha-Longipinene, Lycopene, Menthol, Methyl butyrate, 3-Mercapto-2-Methylpentanal, Mercaptan/Thiols, Beta-Mercaptoethanol, Mercaptoacetic Acid, Allyl Mercaptan, Benzyl Mercaptan, Butyl Mercaptan, Ethyl Mercaptan, Methyl Mercaptan, Furfuryl Mercaptan, Ethylene Mercaptan, Propyl Mercaptan, Thenyl Mercaptan, Methyl Salicylate, Methylbutenol, Methyl-2-Methylvalerate, Methyl Thiobutyrate, Myrcene (Beta-Myrcene), Gamma-Muurolene, Nepetalactone, Nerol, Nerolidol, Neryl acetate, Nonanaldehyde, Nonanoic Acid, Ocimene, Octanal, Octanoic Acid, P-Cymene, Pentyl butyrate, Phellandrene, Phenylacetaldehyde, Phenylethanethiol, Phenylacetic Acid, Phytol, Pinene, Beta-Pinene, Propanethiol, Pristimerin, Pulegone, Quercetin, Retinol, Rutin, Sabinene, Sabinene Hydrate, cis-Sabinene Hydrate, trans-Sabinene Hydrate, Safranal, Alpha-Selinene, Alpha-Sinensal, Beta-Sinensal, Beta-Sitosterol, Squalene, Taxadiene, Terpin hydrate, Terpineol, Terpine-4-ol, Alpha-Terpinene, Gamma-Terpinene, Terpinolene, Thiophenol, Thujone, Thymol, Alpha-Tocopherol, Tonka Undecanone, Undecanal, Valeraldehyde/Pentanal, Verdoxan, Alpha-Ylangene, Umbelliferone, and Vanillin. In some embodiments the one or more terpenes may be in the form of an extract from a plant. In some embodiments the terpene may be in an essentially pure form. In some embodiments the terpene may be in synthetic form. In some embodiments compositions and methods of the invention may comprise 1, 2, 3, 4 or more terpenes.

### Herbal Extracts

Herbal medicine is one of the most commonly sought forms of Complementary and Alternative Medicine (CAM). As used herein “herbal extract” refers to any compound derived from plants or plant material, including medicinal ingredients used in CAM. The clinical use of herbal extracts may follow CAM or traditional methods; the optimum dose and treatment duration varies depending upon the condition treated.

In the United States, herbal medicine is currently used by nearly twenty million Americans⁷⁰, with an annual turnover of more than 1.5 billion dollars and growth of approximately 25% each year⁷¹. According to Hexa Research, the global herbal medicine market was valued at USD 71.19 billion in 2016.⁷² One of the most common conditions for which adults use herbal medicine is pain. For example, it has been estimated that at least 60% of individuals with arthritis pain or other musculoskeletal pain have tried CAM.⁷³ According to the World Health Organization (WHO) 1996 guidelines, herbal medicine comprises active end products that contain underground or aerial parts of either plants or plant materials or a combination of both. Many herbal medicines affect eicosanoid metabolism by inhibiting either both or one of the lipoxygenase and cyclooxygenase (COX) pathways.⁷¹

In addition to pain management, CAM also frequently employs herbal extracts to treat various inflammatory conditions and disorders. Patients with inflammatory conditions who consult CAM providers self-report more intense symptoms than non-users of CAM, and often have other chronic conditions. They do not seem to reject the traditional health care system, but supplement it with CAM, possibly to fulfill needs insufficiently satisfied by traditional health care providers. In 1996-97, among the 3.3 million Canadian adults aged 20 years or older who self-reported arthritis, 22% utilized CAM⁷⁴ in the preceding year.

CAM users tended to be younger and with higher education and household income. They reported more pain, consumed more analgesics, and tended to be more depressed. The coexistence of back or bowel disorders, cancer, sinusitis, or food allergies with arthritis was also related to CAM use. Moreover, CAM users also used more traditional health resources. CAM use in patients with chronic diseases in primary care is associated with perceived quality of care and cultural beliefs. The one year prevalence of CAM use was 22.7%. Patients who were dissatisfied/very dissatisfied with the cost of treatment [odds ratio (OR)=1.79, 95% confidence interval (CI) 1.15-2.82] and waiting time (OR=1.96, 95% CI 1.20-3.19) were more likely to use CAM. Complementary and alternative therapies were popular among patients with chronic pain disorders⁷⁵ surveyed in academic primary care settings. When asked to choose between traditional therapies or CAM, most patients still preferred traditional therapies for pain relief. Fifty-two percent reported current use of CAM for relief of chronic pain. Of the patients that used CAM, 54% agreed that nontraditional remedies helped their pain and 14% indicated that their individual alternative remedy entirely relieved their pain. Vitamin and mineral supplements were the most frequently used CAM modalities.

CAM is frequently used in the management of insomnia which commonly accompanies pain-related disorders. Insomnia is a widespread sleep disorder in the general population, and it is a risk factor for impaired function, the development of other medical and mental disorders, and causes an increase in health care costs. In view of the health hazards of insomnia and the shortcomings of traditional western medicine, CAM may be an effective treatment adjunct or alternative. The safety of CAM for insomnia has been found to be acceptable.⁷⁶ Meanwhile, based on pre-clinical trial, the possible mechanisms of CAM for insomnia were modulation of circadian rhythm, GABA receptor activation, antagonisms of 5-HT receptors, inhibition of glutamate-mediated pathways, and attenuation of inflammation.

Herbal extracts may also be employed in CAM for prevention or treatment of hot flashes, which is often associated with insomnia. Prevalence of hot flashes was 12.5% in premenopause, 79.0% in perimenopause, and 39.3% in postmenopause. Prevalence of chronic insomnia^{77,78,79,80} was reported as 36.5% in premenopause, 56.6% in perimenopause, and 50.7% in postmenopause (P<0.001). Prevalence of symptoms of chronic insomnia increased with the severity of hot flashes, reaching more than 80% in perimenopausal women and postmenopausal women who had severe hot flashes.

Many different types of herbal extracts have been shown to be biologically active. Examples of herbal extracts used for medicinal purposes, such as for treatment of pain and/or inflammation, includes St John's Wort (SJW), ginger, turmeric, capsaicin, thunder god vine, butterbur, feverfew, willow bark, black cohash, common hollyhock, garlic, cayenne, bitter orange, yellow lady's slipper, coca, jasmine, henna, dong quai, evening primrose oil, kava Other herbal extracts used for medicinal purposes include Californian poppy, eucalyptus, chamomile, noni, opium poppy, passion flower, kava, white willow, elderberry, Ashoka tree, blue snakeweed, common chickweed, fenugreek, wheatgrass, valerian, lady's mantle, silverweed, raspberry leaf, hibiscus flower, yarrow, and chasteberry. By way of general background information, a list of herbal extracts used for medicinal purposes for management of inflammation is set forth in Ghasemian et al.⁸¹ and for pain by Jahromi et al.⁸² Further, a list of herbal extracts with anti-inflammatory activity including the name of herb, plant part, chemical constituents, type of study showing in vitro and/or in vivo biological activity and study reference has been reported.^{83,84}

By way of non-limiting examples, a synopsis of the medicinal effects of selected herbal extracts potentially useful for pain management and/or inflammation is set out below.

### St John's Wort

St John's Wort (SJW) is extracted from the flowers and leaves of the plant Hypericum perforatum native to Asia and Europe, which was later introduced to North America by the Europeans. SJW includes at least 10 active constituents but the two principal pharmacological components are hyperforin and hypericin, which are responsible for their beneficial effects.^{85,86} Hypericin can inhibit serotonin, norepinephrine, and dopamine reuptake, weakly inhibits monoamine oxidases (MAOIs) A and B and the crude extracts have a high affinity for gamma-aminobutyric acid (GABA) receptors. This has led to its role as an anxiolytic, sedative, antidepressant, and analgesic⁸⁷. Clinical studies and review articles on St John's Wort and pain management have been reported by Jahromi et al.⁸²

### Ginger

Ginger has uses for muscle pain and swelling, arthritis, headaches, digestive and appetite problems, prevention of motion sickness, postoperative nausea and vomiting, hyperemesis gravidarum, and also cold and bacterial infections due to its anti-oxidant mechanism.^{87,88} Gingerols, especially 6-gingerol, are the active components of ginger. Its anti-inflammatory effects come from inhibiting arachidonic acid metabolism.^{89,90} Based on the available data there is a delayed therapeutic action and therefore it does not help treat acute pain conditions such as exercise-induced muscle pain.^{91,92} Clinical studies and review articles on ginger and pain management have been reported by Jahromi et al.⁸²

### Tumeric

Turmeric has traditionally been used as an antiseptic, anti-inflammatory agent for wound healing as well as an antioxidant and analgesic agent.⁹³ Tumeric contains bioactive curcuminoids such as curcumin. Curcumin can regulate inflammatory cytokines such as interleukin (IL)-1 beta, IL-6, IL-12, Tumor necrosis factor (TNF)-alpha, interferon (IFN) gamma, and associated AP-1, NF-kappa B, and JAK-STAT signaling pathways. With its anti-inflammatory effects, it has been used in autoimmune diseases such as rheumatoid arthritis, inflammatory bowel disease, and multiple sclerosis.⁹⁴ In terms of drug interactions, turmeric supplementation of paclitaxel chemotherapy was found to improve the quality of life and pain scores in breast cancer patients.⁹⁵ Clinical studies and review articles on tumeric and pain management have been reported by Jahromi et al.⁸²

### Capsaicin

Capsaicin is a natural chili pepper extract and its topical application is an established treatment option for various pain conditions.⁹⁶ Intense or repetitive exposure to capsaicin leads to a reversible and selective loss of nociceptive nerve endings. It specifically opens the nonselective cation channel, transient receptor potential cation channel subfamily V member 1 (TRPV1) also known as vanilloid receptor, predominantly found in C-fiber polymodal nociceptors. The influx of cations into the neurons leads to a transient burst of action potentials, leading to a profound burning, stinging, mechanical and thermal hyperalgesia. It is followed by reversible desensitization which can last for several weeks. This effect of capsaicin is referred to as “defunctionalization”. While pain receptors regenerate in 4 to 16 weeks, the pain fibers cannot transmit pain signals, resulting in a long-term decrease in sensitivity of mechanical, thermal, and noxious stimulation.^{97,98} A randomized controlled trial found that supplementing topical diclofenac with capsaicin offered superior pain relief compared to diclofenac alone, but not compared to capsaicin alone.⁹⁹ In fact, topical capsaicin is well-tolerated in combination and no drug interactions have been noted so far. 100

The analgesic effect of capsaicin (the active ingredient in Capsicum frutescens Linn. [Solanaceae]) had been reported, both the ethyl acetate extract of Capsicum frutescens Linn. [Solanaceae] (CFE) and the capsaicin (Fluka Biotechnika-CPF). Capsaicin in both forms (CFE and CPF) produced anti-inflammatory effects that were comparable to diclofenac in the experimental rat model at p<0.05 and it may be concluded that capsaicin has both analgesic and anti-inflammatory properties, comparable with Diclofenac (100 mg/kg).¹⁰¹ Clinical studies and review articles on capsaicin and pain management have been reported by Jahromi et al.⁸²

### Thunder God Vine

Thunder god vine is also known as Tripterygium wilfordii Hook F (TwHF) is a traditional Chinese herb. With its anti-inflammatory, immunosuppressive, and analgesic effects, it has been used for rheumatoid arthritis (RA) joint pain and insect pests.¹⁰² It inhibits the expression of proinflammatory cytokines, proinflammatory mediators, adhesion molecules, and matrix metalloproteinases by lymphocytes, macrophages, synovial chondrocytes, and fibroblasts.¹⁰³ Clinical studies and review articles on thunder god vine and pain management have been reported by Jahromi et al.⁸²

### Butterbur

Butterbur is the root extract of Petasites hybridus, a perennial shrub that was used since ancient times for its medicinal properties such as fever, wound healing, muscle spasm, and migraine prophylaxis. The active agents are likely its sesquiterpenes such as petasin and isopetasin.¹⁰⁴ These effects on pain and neurogenic inflammation may count for its role as an anti-migraine treatment.^{104,105} Clinical studies and review articles on butterbur and pain management have been reported by Jahromi et al.⁸²

### Feverfew

Feverfew constitutes the dried leaves of the weed plant Tanacetum parthenium. Several centuries ago it was used to treat fever, headaches, and inflammation. It was rediscovered in the late 20th century for migraine headaches. Its active agents are the parthenolide within the leaves. It can inhibit serotonin release from white blood cells and platelets, and prevent platelet aggregation. It can also have anti-inflammatory action by inhibiting phospholipase A and prostaglandin synthesis.¹⁰⁴ Clinical studies and review articles on feverfew and pain management have been reported by Jahromi et al.⁸²

### Willow Bark Extract

Willow bark extract is one of the first examples of modern medication development from an herbal drug. It is obtained from the willow tree also known as Salix, and is generally standardized to salicin but may contain other salicylates as well as flavonoids and polyphenols. It has been used for thousands of years for its antipyretic, analgesic, and anti-inflammatory effects.¹⁰⁶ The active agents of willow bark extract inhibit COX-2 mediated release of prostaglandins E2 and the release of interleukin 1ß and tumor necrosis factor-α.¹⁰⁷ Clinical studies and review articles on willow bark and pain management have been reported by Jahromi et al.⁸²

### Black Cohash

Actaea racemosa (AR)¹⁰⁸ also known as Cimicifuga racemosa, is a perennial plant from Ranunculaceae family which was used as traditional remedies in treatment of various condition like rheumatoid muscular pain, headache, inflammation and dysmenorrhea. The analysis on Actaea racemose showed various indications for different plant's extracts. Approximately 131 chemical compounds have been isolated and identified from Actaea racemosa. According to recent studies, the most important chemicals known of the Actaea racemosa are phenolic compounds, chromones, triterpenoids, nitrogen-containing constituents. There are a wide range of pharmacological activities for Black cohosh including attenuating menopausal symptoms.

Althaea rosea, Hollyhock

A flower extract decoction is used to improve blood circulation, for the treatment of constipation, dysmenorrhoea¹⁰⁹, haemorrhages, etc. ARF water extract (ARFW) could dose-dependently increase NO production and cytokines (IL-6 and TNF-α). We also found that ARFW significantly increased the expression of iNOS and COX-2 proteins in RAW264.7 cells. Althaea officinalis¹¹⁰ contained pectins 11%, starch 25-35%, mono-, and di-saccharide, saccharose 10%, mucilage 5%, flavonoids (Hypolaetin-8-glucoside, isoquercitrin, kaempferol, caffeic, pcoumaric acid), coumarins, scopoletin, phytosterols, tannins, asparagine and many amino acids. The previous studies showed that Althaea officinalis possessed antimicrobial, antiinflammatory, immunomodulatory, demulcent, soothing, antitussive and many other pharmacological effects. Althaea rosea contained high molecular weight acidic polysaccharides (1.3 to 1.6 million Dalton) known as mucilages which are found in flowers and leaves.

Lawsonia inermis, Henna

The terpene, β-ionone¹¹¹ is largely responsible for the pungent odour of the essential oil isolated from the flowers. In addition to other volatile terpenes, some non-volatile terpenoids, a single sterol, two alkaloids and two dioxin derivatives have also been isolated from the plant. Lawsonia inermis-extracted oil is known for therapeutic properties, especially wound healing. L. inermis extract treatment decreased the elevation of inflammatory markers including IL1-β, and TNF-α in the spinal cord of CCI rats. These results indicated that L. inermis has potential neuroprotective effects against CCI induced neuropathic pain due to its anti-oxidant, and anti-inflammatory effects.¹¹² Lawsonia inermis-extracted oil is known for therapeutic properties, especially wound healing.¹¹³ The quality and content of the fatty acids in the oil were determined and histological and chromatic assessment findings revealed healing in the oil-treated group a full reepithelialization with reappearance of skin appendages and well-organized collagen fibers without any inflammatory cells. This might be due to a synergistic effect of the phytoconstituents present in the oil.

Angelica sinensis, Dong quai

The dry root of Angelica sinensis (Oliv.) Diels, also known as “female ginseng”, is a popular herbal drug amongst women, used to treat a variety of health issues and cardiovascular diseases. The detailed molecular mechanism for anti-inflammatory effects of Angelica sinensis root water extract (ASW) suggests that ASW exerts an anti-inflammatory effect on LPS-induced RAW 264.7 via NO-bursting/calcium-mediated JAK-STAT pathway.¹¹⁴ The main chemical constituents of Angelica roots include ferulic acid, Z-ligustilide, butylidenephthalide and various polysaccharides. Among these compounds, ferulic acid exhibits many bioactivities especially anti-inflammatory and immunostimulatory effects¹¹⁵; Z-ligustilide exerts anti-inflammatory, anti-cancer, neuroprotective and anti-hepatotoxic effects; n-butylidenephthalide exerts anti-inflammatory, anti-cancer and anti-cardiovascular effects.

Morinda citrifolia

In Jamaican folklore practice, noni juice (Morinda citrifolia, noni, Indian mulberry, duppy soursop) is used to treat diverse conditions, including hypertension, diabetes mellitus, bronchial asthma, rheumatoid arthritis, some cancers, and sexual dysfunction The effect of the fruit juice (oral and IP) may reflect a more extensive action against several inflammatory mediators such as histamine, hydroxytryptamine, bradykinin, prostaglandin¹¹⁶ and nitric oxide, all of which are reported to be involved in carrageenan-induced edema. Absence of toxic effects when the juice extract was given acutely and chronically via the oral route in rats. The anti-inflammatory effects of noni juice were investigated in vitro by: measuring its effect on nitric oxide and prostaglandin E2 production by activated macrophages, evaluating its inhibitory activities on cyclooxygenase (COX)-1 and -2 and in vivo on a carrageenan-induced paw oedema model in rats.¹¹⁷ Several polyphenols belonging to the coumarin, flavonoid and phenolic acid groups, and two iridoids were identified. It directly inhibited cyclooxygenase COX-1 and COX-2 activities and inhibited the production of nitric oxide (NO) and prostaglandins E2 (PGE2) in activated J774 cells, in a dose dependent manner. Fatty Acid Esters can be isolated.¹¹⁸

Evening Primrose Oil, (Oenothera biennis)

Oenothera biennis with the common name of “evening primrose” contains a valuable fixed oil with the commercial name of EPO. Evening primrose oil has two types of omega-6-fatty acid including linoleic acid (60%-80%) and γ-linoleic acid (8%-14%). Essential fatty acids are considered as essential compounds for body health, especially among women.¹¹⁹ On current evidence EPO is of little value in the management of premenstrual syndrome.¹²⁰ Evening primrose oil, which includes GLA, is one of the most popular of many treatments available for PMS. Oral 180 mg/day of GLA for three luteal phases can be effective for treating the symptoms of PMS, and that GLA and DGLA in plasma phospholipid.

Velvet bean (Kapikacchu, Mucuna pruriens)

Mucuna pruriens (Fabaceae) is an established herbal drug used for the management of male infertility, nervous disorders, and also as an aphrodisiac. M. pruriens has been shown to have anti-parkinson and neuroprotective effects, which may be related to its anti-oxidant activity. In addition, anti-oxidant activity of M. pruriens has been also demonstrated in vitro by its ability to scavenge DPPH radicals and reactive oxygen species. In this review the medicinal properties of M. pruriens are summarized, taking in consideration the studies that have used the seeds extracts and the leaves extracts.¹²¹ Factors include polyphenols, trypsin inhibitors, phytate, cyanogenic glycosides, oligosaccharides, saponins, lectins, and alkaloids. Polyphenols (or tannins) are able to bind to proteins, thus lowering their digestibility. Phenolic compounds inhibit the activity of digestive as well as hydrolytic enzymes such as amylase, trypsin, chymotrypsin, and lipase. Recently, phenolics have been suggested to exhibit health related functional properties such as anti-carcinogenic, anti-viral, anti-microbial, anti-inflammatory, hypotensive, and anti-oxidant activities. Fatty acid profiles reveal that lipids are a good source of the nutritionally essential linoleic and oleic acids. Linoleic acid is evidently the predominant fatty acid, followed by palmitic, oleic, and linolenic acids. Apart from high protein and starch content, these beans contain (l-Dopa) 3,4-dihydroxy-l-phenylalanine, which exhibits several medicinal properties.¹²² Mucuna seeds are known to produce the unusual nonprotein amino acid 3-(3,4 dihydroxyl phenyl)-l-alanine (l-Dopa), a potent neurotransmitter precursor that is, at least in part, believed to be responsible for the toxicity of Mucuna seeds (Cilia et al. 2017). l-Dopa is an intermediary product in the enzymatic synthesis of dopamine from l-tyrosine. Dopamine regulates functions in the brain (neurotransmitter), heart (an inotropic increase of cardiac output), vascular system (vessel dilator), and kidney (diuretic) (Grover et al. 2001).

### Kava Kava

Kava extract preparations were once used as herbal drugs to treat anxiety in Europe. Recent studies suggest that kava and its key phytochemicals have anti-inflammatory and anticancer effects, in addition to the well-documented neurological benefits. relaxant and medicinal effects as a pain reliever, muscle relaxant, and as a remedy for anxiety, nervousness and insomnia. daily oral dose range of 20-300 mg kavalactones, particularly for mild and moderate anxiety.¹²³ The mechanisms of action¹²⁴ proposed for kava kava include decreased levels of glutamate, an excitatory neurotransmitter, activation of dopaminergic neurons, interaction with GABA receptors, direct action on muscles leading to relaxation, elevation of dopamine and serotonin levels via inhibition of monoamine uptake, and cellular actions similar to mood stabilizers.

Rhodiola rosea

Rhodiola rosea L., a worldwide botanical adaptogen, has been confirmed to possess protective effects of inflammatory injury for many diseases, including cardiovascular diseases, neurodegenerative diseases, diabetes, sepsis, and cancer.¹²⁵ R. rosea extracts and its purified constituent, salidroside, has been shown to produce a variety of mediator interactions with several molecular networks of neuroendocrine-immune and neurotransmitter receptor systems likely to be involved in the pathophysiology of depression.¹²⁶

Calamintha nepeta (L.) Savi

Calamintha nepeta (L.) Savi is an aromatic herb with a mint-oregano flavor, used in the Mediterranean areas as a traditional medicine. It has an extensive range of biological activities, including antimicrobial, antioxidant and anti-inflammatory, as well as anti-ulcer and insecticidal properties. It has been widely used against insomnia, depression, convulsion and cramps and for the treatment of respiratory and gastroenteric diseases. The major components in the Savi oils generally belong to the C-3 oxygenated p-menthanes such as PUL, menthone, isomenthone, and piperitone and piperitenone with their oxides or, more rarely, C-6 oxygenated p-menthane compounds such as carvone. (R)-5-Methyl-2-(1-methylethylidine) cyclohexanone is a monoterpene ketone, known as PUL. PUL is practically insolubile in water but miscible with ethanol, diethyl ether and chloroform. Two enantiomers occur in nature, the R-(+)-form being the most abundant in the EO (Essential Oil) PUL has been given Generally Recognized as Safe (GRAS) status by the United States Food and Drug administration since 1965, there are currently no limits in the area of medicinal products. Its concentration in cosmetic formulations should not exceed 1%.¹²⁷ It was found that this terpene blocked prostaglandin and other inflammatory mediator's formation in diarrhea which may in part explain its antisecretory effect. The antinociceptive properties of this monoterpene were assessed using several pain models. Chemical nociception induced in the first and second phase of the subplantar formalin test was significantly inhibited by PUL and was not blocked by naloxone. The results suggest that PUL is a psychoactive compound and has the profile of an analgesic drug.¹²⁸ The toxicological studies of PUL and its metabolites point to the need for precaution in exposure to it, thereby limiting its use in food products (very hepatotoxic).¹²⁴

Vitis vinifera

Seven stilbenoids from Stilbenoid-Rich Root Extract of including resveratrol, piceatannol, trans-ε-viniferin, ampelopsin A, miyabenol C, r-2-viniferin=vitisin A, and r-viniferin=vitisin B were identified in the root extract of Vitis vinifera. The root extract of Vitis vinifera scavenged DPPH, hydroxyl, galvinoxyl, and superoxide free radicals.¹²⁹ Accordingly, a protection against hydrogen peroxide-induced DNA damage was observed in cultured cells. Furthermore, Nrf2 and its target genes HO-1 and γ-GCS as well as PON1 were induced by Vitis vinifera root extract. Moreover, the root extract downregulated proinflammatory gene expression including IL-1B and INOS in cultured macrophages.¹²⁹ Prior studies suggest free radical scavenging and cellular antioxidant and anti-inflammatory activities of the Vitis vinifera root extract in vitro.

Moringa oleifera

The Moringa plant provides zeatin, quercetin, beta-sitosterol, caffeoylquinic acid and kaempferol act as cardiac and circulatory stimulants, possess antitumor, antipyretic, antiepileptic, antiinflammatory, antiulcer, antispasmodic, diuretic, antihypertensive, cholesterol lowering, antioxidant, antidiabetic, hepatoprotective, antibacterial and antifungal activities. Moringa oleifera seeds are a promising resource for food and non-food applications, due to their content of monounsaturated fatty acids with a high monounsaturated/saturated fatty acids (MUFA/SFA) ratio, sterols and tocopherols, as well as proteins rich in sulfated amino acids. MO leaves are becoming the best phytomedicine to reduce hypertension, which are naturally known as angiotensin-1converting enzyme (ACE), acetylcholinesterase, arginase.¹³⁰ 2% and 4% of Moringa leaf and seed inclusive diets for 14 days, could be of great benefit toward the management of ED (Erectile Dysfunction) caused by diabetes, as evidenced by the expression of some ED-related biomarkers in the penile tissue of diabetic male rats.¹³¹

### Antioxidants

Antioxidants are man-made or natural substances that may prevent or delay some types of cellular damage by counteracting oxidative stress.²² In the context of a biological system, oxidative stress refers to an imbalance between the production of oxidants, reactive species derived from oxygen and nitrogen, and antioxidant defenses that may cause damage to macromolecules in the biological system.^{23,24} Damage caused by oxidants is known as oxidative damage. Antioxidants are defined as substances that, when present at low concentrations compared to those of an oxidizable substrate, delay or prevent oxidation of the oxidizable substrate.

Oxidative stress is thought to play a role in a variety of diseases including, but not limited to, cancer, cardiovascular diseases, diabetes, Alzheimer's disease, Parkinson's disease, and eye diseases such as cataracts and age-related macular degeneration.^{25,26,27}

Although the direct link between oxidative stress and pathology of painful conditions, such as complex regional pain syndrome and neuropathy, is not fully established, it is hypothesized that oxidative stress exacerbates inflammation and neuropathy that contributes to development of pain signals. Although supplementation with antioxidants is promoted for pain management, clinical research on the impact of dietary antioxidants on inflammation is inconclusive.

Examples of antioxidants include vitamin C and vitamin E, selenium, and carotenoids, such as beta-carotene, lycopene, lutein, and zeaxanthin.

Resveratrol, a powerful antioxidant and anti-inflammatory component, is found in grapes, wine, grape juice, peanuts, cocoa, blueberries, bilberries, and cranberries.²⁸ Despite encouraging data in preclinical models of pain, the clinical evidence is not conclusive about the superiority of resveratrol over placebo in reducing endometriosis pain.²⁹

Polydatin (PLD) (3,5,4-dihydroxystylate-3-o-β-d-glucopiranoside) is a resveratrol glucoside in which the glycoside group is bound in the C-3 position as a substitute for a hydroxyl group. It is the predominant form in which resveratrol is present in nature. It has been suggested that glucoside can be hydrolyzed to resveratrol in the duodenum or colon.³⁰

In some embodiments, the compositions and methods of the invention may include or use one or more of the following antioxidants: Ascorbic acid, Vitamin A, Vitamin E, Lipoic acid, Masoprocol, Pramipexole, Nitric Oxide, Allopurinol, Pentoxifylline, Melatonin, Dimethyl sulfoxide, Probucol, 3,4-Dihydroxycinnamic Acid, Resveratrol, 3-hydroxyanthranilic acid, Dihydrolipoic Acid, p-Coumaric acid, Quercetin, AEOL-10150, Transcrocetinate, Acetylcysteine, Nicaraven, Lodoxamide, Ferulic acid, Uric acid, Idebenone, Chromic chloride, Thiosulfuric acid, Mequinol, Hydroquinone, Selenic acid, Selenium, Lycopene, Tocopherol, Rebamipide, Allicin, Anisodamine, Epigallocatechin gallate, Bucillamine, Edaravone, Tempol, Propyl Gallate, Apocynin, Tocotrienol, Hydroxytyrosol, Acteoside, Tirilazad, Chromanol, alpha-Tocopherol succinate, alpha-Tocopherol acetate, Sodium bisulfite, Tocopherylquinone, N,N′-diphenyl-1,4-phenylenediamine, 4-(Isopropylamino)diphenylamine, Turmeric, Calcium ascorbate, Kojic acid, Ebselen, Dexpramipexole, gamma-Tocopherol, Carvedilol, 2,5-di-tert-butylhydroquinone, Cyclo(his-pro), Tricetin, t-Butylhydroquinone, Droloxifene, Maritime pine extract, Garlic oil, Baicalein, Silibinin, Vitis vinifera seed, D-alpha-Tocopherol acetate, Sodium ascorbate, Magnesium ascorbate, Ferrous ascorbate, Bisphenol A, Acetylcysteine zinc, Avasopasem manganese, Morin, Tyrosol, Butylated hydroxytoluene, and Rosmarinic acid.

In some embodiments the one or more antioxidants may be in the form of an extract from a plant. In some embodiments the antioxidant may be in an essentially pure form. In some embodiments the antioxidant may be in synthetic form. In some embodiments compositions and methods of the invention may comprise 1, 2, 3, 4 or more antioxidants.

### Prostaglandins

In some embodiments, the compositions and methods of the invention may act on prostaglandins and/or prostaglandin biosynthetic pathways involving cyclooxygenase (COX) enzymes.

Prostaglandins are physiologically active lipid compounds derived from arachidonic acid. They both sustain homeostatic functions and mediate pathogenic mechanisms, including the inflammatory response. These compounds are generated from arachidonate by the action of COX isoenzymes, including Cox-1 and Cox-2, and their biosynthesis is blocked by nonsteroidal anti-inflammatory drugs (NSAIDs). Prostaglandins may function in both the promotion and resolution of inflammation.^{31,132,133,134}

Examples of prostaglandins include prostaglandin 12 (prostacyclin; PGI2), prostaglandin D2 (PGD2), prostaglandin E2 (PGE2), and prostaglandin F2α (PGF2α).³¹

### Pharmaceutical Dosage Forms, Delivery Systems and Methods of Use

The present disclosure encompasses various pharmaceutical dosage forms, delivery systems and methods of use. As used herein “dosage form” refers to any formulation or preparation suitable for administration as a pharmaceutical product. A dosage form may include active pharmaceutical ingredient(s) (APIs) and inactive components. In some embodiments compounds and compositions comprising at least one N-acylethanolamine (NAE) and at least one of a cannabinoid, terpene, herbal extract or antioxidant, or pharmaceutically acceptable salts or derivatives thereof, may be formulated in a dosage form with suitable pharmaceutically acceptable carriers, such as diluents, fillers, salts, buffers, stabilizers, solubilizers, etc. The dosage forms may be configured for different routes of administration, such as forms suitable for oral, rectal, nasal, topical (including oro-mucosal such as buccal and sublingual), vaginal or parenteral (including intramuscular, subcutaneous and intravenous) administration or in a form suitable for administration by inhalation or insufflation.

The dosage forms may contain other pharmaceutically acceptable excipients for modifying conditions such as pH, osmolarity, taste, viscosity, sterility, lipophilicity, solubility etc. The choice of diluents, carriers or excipients will depend on the desired dosage form, which may in turn be dependent on the intended route of administration to a patient.

Suitable dosage forms include, but are not limited to, solid dosage forms, for example tablets, capsules, powders, dispersible granules, cachets and suppositories, including sustained release and delayed release formulations. Powders and tablets will generally comprise from about 5% to about 70% active pharmaceutical ingredients. For preparing pharmaceutical compositions described herein, pharmaceutically acceptable carriers can be either solid or liquid. Solid form preparations include powders, tablets, pills, capsules, cachets, suppositories, and dispensable granules. A solid carrier can be one or more substances which may also act as diluents, flavouring agents, solubilizers, lubricants, suspending agents, binders, preservatives, tablet disintegrating agents, or an encapsulating material.

Suitable liquid dosage forms include solutions, suspensions and emulsions. Liquid form preparations may be administered by intravenous, intracerebral, intraperitoneal, parenteral or intramuscular injection or infusion. Sterile injectable formulations may comprise a sterile solution or suspension of the active agent in a non-toxic, pharmaceutically acceptable diluent or solvent. Liquid dosage forms also include solutions or sprays for intranasal, buccal or sublingual administration. In some embodiments dosage forms may comprise oro-mucosal sprays, sublingual drops and intranasal formulations. Aerosol preparations suitable for inhalation may include solutions and solids in powder form, which may be combined with a pharmaceutically acceptable carrier, such as an inert compressed gas.

Also encompassed are dosage forms for transdermal administration, including creams, lotions, aerosols and/or emulsions and topical (including oro-mucosal such as buccal and sublingual) administration. These dosage forms may be included in transdermal patches of the matrix or reservoir type, which are generally known in the art.

Pharmaceutical preparation doses (for example for at least one N-acylethanolamine (NAE) in combination with at least one of a cannabinoid, terpene, antioxidant or herbal extract) may be conveniently prepared in unit dosage form, according to standard procedures of pharmaceutical formulation.

The quantity of active compound(s) per unit dose may be varied according to the nature of the active compound and the intended dosage regime. Generally an effective amount shall be used, which may be within the range of from 0.01 mg to 5000 mg, preferably 0.01-4000 mg, 0.1-3000 mg, 1-2500 mg, 5-1000 mg, or 10-300 mg per unit dose (for the at least one N-acylethanolamine (NAE) and at least one of a cannabinoid, terpene, antioxidant, or herbal extract). The effective amount may depend upon the means of application, such as creams, sprays, tablets or patches. For example, higher loading of active pharmaceutical ingredient(s) may be used in the case of creams and sprays compared to vaginal films.

Generally, the weight ratio of the at least one of a cannabinoid, terpene, antioxidant, or herbal extract to the at least one N-acylethanolamine (NAE) is decided by considering the properties of each constituent to be combined, the properties of the drug combination and the symptoms of the patient. In some embodiments the weight ratio is in the range of 1 part by weight of the at least one cannabinoid, terpene, antioxidant or herbal extract to about 0.01 to about 500 parts by weight of the at least one N-acylethanolamine (NAE); in other embodiments the weight ratio is in the range of 1 part by weight of the at least one cannabinoid, terpene, antioxidant or herbal extract to about 0.1 to about 100 parts by weight of the N-acylethanolamine (NAE).

In some embodiments compounds and compositions of the invention may be formulated for administration in one or more of the following dosage forms: a tablet, a liquid dosage form, a hard gelatin capsule, a soft gelatin capsule, gel capsules, a non-gelatin capsule, an HPMC capsule, an inhalant, an injectable, medical patches, topicals, creams, varnishes, sublingual oils, sprays, edibles, a transdermal, a buccal, microneedles, a sublingual patches, a rectal or a vaginal suppository, tampons, cigarettes, vaporizer liquids, nasal preparations, preparations containing micro and/or nano-emulsions, and preparations containing micro and/or nano-particles. In some embodiments compounds and compositions may be formulated for administration in a dosage form suitable for sustained release, or extended release, or a combined sustained release and extended release dosage forms, or in an immediate release dosage forms, or a combined sustained release and immediate release dosage forms. Further, compounds and compositions may be formulated for administration in combination with one or more other medications or dietary supplements. In some embodiments the formulations and methods of delivery may be configured to increase bioavailability, for example due to route of administration, water solubility and/or dosage of active pharmaceutical ingredients.

As used herein “delivery systems” refers to technologies configured for the targeted delivery and/or controlled release of therapeutic agents, such as the compounds, compositions and dosage forms of the invention. In some embodiments the compounds and compositions of the invention can be delivered using gels, gel-forming films and/or gel-forming tablets. Such embodiments can be formulated and optimized as will be understood by a person skilled in the art. By way of example, a polymer or combination of polymers may be selected as the carrier, such as sodium carboxymethylcellulose (CMC), polyvinyl alcohol (PVA) and hyaluronic acid (HA). The film forming technique may, for example, be solvent casting or electrospinning. The ratio of the polymers may be selected to optimize the mucoadhesive, residence time, flexibility or other properties of the films. The durability of the films in liquid may be enhanced by post-casting modifications, such as crosslinking. The flexibility of the films may be optimized by adding plasticizers, such as glycerol and polyethylene glycol. The release profile of the active ingredients may also be optimized. In some embodiments, multi-layer films may be formulated.

Further in regard to possible polymers, sodium carboxymethylcellulose (CMC) is a cellulose derivative with carboxymethyl groups bound to some of the hydroxyl groups of the cellulose backbone. Following addition to an aqueous phase, this linear polymer undergoes swelling prior to dissolution and forms a gel, which forms adhesive interactions with biological substrates. CMC mucoadhesive properties provide certain advantages for drug delivery (e.g., improved location on and prolonged residence time), which lead to extensive application of this polymer in drug delivery systems targeting buccal cavity, nasal, vaginal and gastrointestinal tracts, and the eye. Polyvinyl alcohol (PVA) is a water soluble polymer whose water solubility depends on its degree of hydrolysis, molecular weight, and tendency to hydrogen bond in aqueous solutions. In some embodiments, a combination of CMC and PVA may be selected as carrier polymers. In some embodiments, films may also optionally include anti-inflammatory and/or antiseptic agents. In one particular embodiment, a drug delivery vehicle formulation formed by a solvent casting technique may comprise a single layer gel-forming film containing 5% poloxamer 188, 10% glycerol, 35% CMC, 35% PVA. The remainder of the formulation (15% of W/W) will be active pharmaceutical ingredients (APIs)

In regard to formulation of gel-forming tablets, example carrier polymers include polyacrylic acid (carbomer) and hydroxypropyl methylcellulose (HPMC). Carbomer is a synthetic high-molecular weight, crosslinked polyacrylic acid polymer that has excellent mucoadhesive and hydrogel forming properties. HPMC is a film-forming commonly used in the pharmaceutical industry as a rate-controlling polymer for sustained-release dose forms. In one particular example embodiment, a drug delivery system comprising single layer pressed tablets may comprise the features of Formulation A or B set forth in the following table.

| | API | Carbomer | HPMC |
|---|---|---|---|
| Formulation | % | % | % |
| A | 20 | 37 | 43 |
| B | 25 | 37 | 38 |

In another particular example embodiment, a drug delivery system may comprise a double layer pressed tablets formulation. In this two-layer tablet example, 10 mg of polymer and drug mixture of the top layer (as indicated in the table below) were added to the press machine. Subsequently 40 mg of the bottom layer (20% API, 37% carbomer and 43% HPMC) was added to the press machine reservoir and pressed. These two-layer tablets strongly and instantly adhere to a mucosal membrane of a patient, such as the buccal mucosa.

| | API | Carbomer | HPMC |
|---|---|---|---|
| Formulation | % | % | % |
| Top layer A | 50 | 25 | 25 |
| Top layer B | 45 | 27.5 | 27.5 |
| Bottom | 20 | 37 | 43 |
| layer | | | |

Fast dissolving gel-forming films can be deployed as a means for fast or sustained delivery of pharmaceutically active agents across mucosal membranes or other tissue targets. For example, the films may be configured for administration to the vaginal mucosa for targeted treatment of symptoms related to dysmenorrhea; or to the rectal mucosal for targeted treatment of symptoms related to haemorrhoidal pain; or to vulva for targeted treatment of symptoms relating to vulvodynia or vulvar pain. In some embodiments the films may be fast dissolving gel-forming films.

In one particular example embodiment, a drug delivery system may comprise a hydrophilic vaginal or rectal suppository comprising the features of Formulation A to F set forth in the following table. This formulation dissolves in the bodily fluids upon insertion into the vaginal and rectal cavity and releases the APIs.

| | | POLYPEG SUPPOSITORY |
|---|---|---|
| Formulation | API % | BASE |
| A | 30 | 70 |
| B | 25 | 75 |
| C | 20 | 80 |
| D | 15 | 85 |
| E | 10 | 90 |
| F | 5 | 95 |

In another particular example embodiment, a drug delivery system vehicle formulation may comprise a vaginal or rectal suppository. In this suppository example, 2 gr of POLYPEG SUPPOSITORY BASE 0746 (MEDISCA Products) were heated up in a water bath at 80 degrees to melt and 25 mg of CBD, 25 mg of CBG and 250 mg of PEA were added to the base (87% POLYPEG SUPPOSITORY base, 11% PEA, 1% CBD, 1% CBG). The base was heated to 85-90 degrees centigrade so all APIs dissolve in the base. Then the base was poured in suppository molds and left at room temperature to harden.

In another particular example embodiment, a drug delivery system vehicle formulation may comprise a vaginal or rectal suppository. in this suppository example, 2 gr of POLYPEG SUPPOSITORY BASE 0746 (MEDISCA Products) were heated up in a water bath at 80 degrees to melt and 12.5 mg of CBD, 12.5 mg of CBG and 250 mg of PEA were added to the base (88% POLYPEG SUPPOSITORY base, 11% PEA, 0.5% CBD, 0.5% CBG). The base was heated to 85-90 degrees centigrade so all APIs dissolve in the base. Then the base was poured in suppository molds and left at room temperature to harden.

In another particular example embodiment, a drug delivery system vehicle formulation may comprise a vaginal or rectal suppository. In this suppository example, 2 gr of POLYPEG SUPPOSITORY BASE 0746 (MEDISCA Products) were heated up in a water bath at 80 degrees to melt and 50 mg of CBD, 50 mg of CBG and 250 mg of PEA were added to the base (85% POLYPEG SUPPOSITORY base, 10.5% PEA, 2% CBD, 2% CBG). The base was heated to 85-90 degrees centigrade so all APIs dissolve in the base. Then the base was poured in suppository molds and left at room temperature to harden.

In another particular example embodiment, a drug delivery system vehicle formulation may comprise a vaginal or rectal suppository. In this suppository example, 2 gr of POLYPEG SUPPOSITORY BASE 0746 (MEDISCA Products) was heated up in a water bath at 80 degrees to melt and 100 mg of CBD, 100 mg of CBG and 250 mg of PEA were added to the base (81.6% POLYPEG SUPPOSITORY base, 10.2% PEA, 4% CBD, 4% CBG). The base was heated to 85-90 degrees centigrade so that all APIs were dissolved in the base. Then the base was poured in suppository molds and left at room temperature to harden.

In one particular example embodiment, a drug delivery system may comprise a hydrophobic vaginal or rectal suppository comprising the features of Formulation A to F set forth in the following table. This formulation melts upon insertion into the vaginal and rectal cavity and releases the APIs.

| Formulation | API % | Cocoa Butter |
|---|---|---|
| A | 30 | 70 |
| B | 25 | 75 |
| C | 20 | 80 |
| D | 15 | 85 |
| E | 10 | 90 |
| F | 5 | 95 |

In another particular example embodiment, a drug delivery system vehicle formulation may comprise a vaginal or rectal suppository. In this suppository example, 2 gr of Cocoa Butter was heated up in a water bath at 60 degrees to melt and 12.5 mg of CBD, 12.5 mg of CBG and 250 mg of PEA were added to the base (88% Cocoa Butter, 11% PEA, 0.5% CBD, 0.5% CBG). The base was heated to 60-80 degrees centigrade so that all APIs were dissolved in the base. Then the base was poured in suppository molds and left at room temperature to harden.

In another particular example embodiment, a drug delivery system vehicle formulation may comprise a vaginal or rectal suppository. In this suppository example, 2 gr of Cocoa Butter was heated up in a water bath at 60 degrees to melt and 50 mg of CBD, 50 mg of CBG and 250 mg of PEA were added to the base (85% Cocoa Butter, 10.5% PEA, 2% CBD, 2% CBG). The base was heated to 60-80 degrees centigrade so that all APIs were dissolved in the base. Then the base was poured in suppository molds and left at room temperature to harden.

In another particular example embodiment, a drug delivery system vehicle formulation may comprise a vaginal or rectal suppository. In this suppository example, 2 gr of Cocoa Butter was heated up in a water bath at 60 degrees to melt and 100 mg of CBD, 100 mg of CBG and 250 mg of PEA were added to the base (81.6% Cocoa Butter, 10.2% PEA, 4% CBD, 4% CBG). The base was heated to 60-80 degrees centigrade so that all APIs were dissolved in the base. Then the base was poured in suppository molds and left at room temperature to harden.

The suppositories can be deployed as a means for fast delivery of pharmaceutically active agents across mucosal membranes or other tissue targets. For example, the suppository may be configured for administration to the vaginal mucosa for targeted treatment of symptoms related to dysmenorrhea, endometriosis, or gynecological cancers (such as, cervical, ovarian, uterine, vaginal, vulvar or fallopian tube); or to the rectal mucosal for targeted treatment of symptoms related to haemorrhoidal pain; or to vulva for targeted treatment of symptoms relating to vulvodynia or vulvar pain.

In some embodiments the compounds and compositions of the invention may be used in a drug delivery system that comprises or is used in conjunction with an applicator or other deployment instrument suitable for the target site. For example, in the case of treatment of pelvic pain or pain-related symptoms, the drug delivery system may comprise or be used in association with an applicator/instrument such as a TENS machine, vaginal massager, vibrator, vaginal dilator, dildo or the like. Further, a drug delivery system may used in conjunction with methods to improve delivery of active agents, including electrical methods (iontophoresis, electroporation); mechanical methods (massage, heat pads, microneedle, puncture, perforation, abrasion, needleless injection, suction, stretching); ultrasound; magnetophoresis; radio frequency; and laser and photomechanical waves. In some embodiments the applicator/instrument may employ chemical, mechanical, sonic, light and/or other stimuli to enhance the receptiveness of the tissue at the target site to treatment, for example to increase the penetration or bioavailability of compounds and compositions of the invention at the target site.

The compounds and compositions of the invention (optionally deployed in dosage forms and/or drug delivery systems) may be used for treating and/or preventing pain or pain-related symptoms in a patient. In some embodiments methods of treatment or prevention may comprise administering to the patient at least one N-acylethanolamine (NAE) in combination with at least one of a cannabinoid, terpene, antioxidant or herbal extract. In some embodiments the at least one of a cannabinoid, terpene, antioxidant or herbal extract is administered separately, sequentially or simultaneously to the at least one N-acylethanolamine. In some embodiments the pain may be associated with one or more of primary or secondary dysmenorrhea, endometriosis, pelvic pain, postpartum pain, labor contractions, polycystic ovary syndrome (POCS), oligomenorrhea, hypermenorrhea, metrorrhagia, pain associated with sexual intercourse, abdominal pain, sexual pain disorder, dyspareunia, breast pain and/or mastalgia, pain associated hormonal dysfunction, hormonal imbalance, hormonal changes, muscle pain, migraine, headache, radicular pain and/or post-surgery pain, muscle spasm, cramps, bone pain, joint pain, arthritis, osteoarthrosis, dental and gum pain, stomach ulcers-related pain, gallbladder disease-related pain, central pain syndrome, sports trauma, vulvar pain, haemorrhoidal pain, chronic pain disorder (nociceptive pain, neuropathic pain, chronic back or leg pain, painful neuropathies, complex regional pain syndrome), neurological pain or acute pain. In some embodiments the pain-related symptoms may include nausea, fatigue, bowel symptoms, hot flashes, facial flushing, night sweat, osteoporosis, indigestion, digestive problems, intestinal disorders, constipation, diarrhea, bladder symptoms, and one or more symptoms caused by pelvic muscle pain or spasm, chronic pelvic pain, and pain related to sexual intercourse.

Administration of the compounds and compositions of the invention may fully or partially treat and/or prevent pain or pain-related symptoms in a patient. In some embodiments administration of the compounds and compositions may alleviate pain or pain-related symptoms and/or help the patient manage pain or pain-related symptoms and/or reduce pain or pain-related symptoms from a severe state to a less severe state.

In some embodiments the compounds and compositions of the invention may be used for treating and/or preventing inflammatory disorders in a patient. In some embodiments the inflammatory disorders may include rheumatoid arthritis, systemic lupus erythematosus (lupus), inflammatory bowel disease (IBD), multiple sclerosis (MS), Type 1 diabetes mellitus, Type 2 diabetes mellitus, Guillain-Barre syndrome, chronic inflammatory demyelinating polyneuropathy, psoriasis, allergy, asthma, atherosclerosis, atopic dermatitis, autoinflammatory syndrome, and Crohn's disease.

In some embodiments the compounds and compositions of the invention may be used for treating and/or preventing psychological disorders in a patient. In some embodiments the psychological disorders may relate to depressive symptoms and/or anxiety relating to the management of chronic pain. In some embodiments the psychological disorders may include one or more of depression, psychotic disorder, schizophrenia, schizophreniform disorder (acute schizophrenic episode); schizoaffective disorder; bipolar I disorder (mania, manic disorder, manic-depressive psychosis); bipolar II disorder; major depressive disorder with psychotic feature (psychotic depression); delusional disorders (paranoia); shared psychotic disorder (shared paranoia disorder); brief psychotic disorder (other and unspecified reactive psychosis); psychotic disorder not otherwise specified (unspecified psychosis); paranoid personality disorder; schizoid personality disorder; schizotypal personality disorder, anxiety disorder, panic disorder, panic attacks, agoraphobia, attention deficit syndrome, premenstrual dysphoric disorder (PMDD), premenstrual tension, premenstrual syndrome (PMS), irritability, lack of focus, lack of alertness, and poor day function.

In some embodiments the compounds and compositions of the invention may be used for treating and/or preventing of spasticity, muscle cramping or spasticity pain-related symptoms in a patient.

In some embodiments the compounds and compositions of the invention may be used for improving sleep duration or sleep quality in a subject.

In some embodiments the compounds and compositions of the invention may be used to inhibit Cox-1 or Cox-2 enzymes and/or to inhibit prostaglandin production.

As indicated above, in some embodiments the compounds and compositions of the invention may be administered together with one or more pharmaceutically acceptable carriers, excipients or diluents. In some embodiments the compounds and compositions of the invention may be administered in combination with one or more other medications or dietary supplements.

### EXAMPLES

Certain embodiments are further described with reference to the following examples, which are intended to be illustrative and not limiting in nature.

### Example 1—Vaginal Suppository for Targeted Treatment of Symptoms Related to Dysmenorrhea or Menstrual Pain

A female patient, aged 36, with a history of moderate to severe menstrual pain (VAS 6-7/10) and cramps was advised to take vaginal suppositories containing 50 mg CBD:50 mg CBG:250 mg PEA at the first sign of cramps or lower back pain associated with her next menstrual period. This dose was to be repeated every 8 hours. No other medication was to be taken.

The patient reported severe abdominal and lower back pain (VAS 7/10) and cramps at day one of her subsequent menstrual period (bleeding). After enduring the pain for 30 minutes and recording that the severity of pain and cramps was increasing, the patient inserted the above-mentioned vaginal suppository (50 mg CBD:50 mg CBG:250 mg PEA) on the first day of the bleeding (light bleeding). Within 20 minutes, the pain began to subside and by 45 minutes was lessened to VAS 2 and the cramps were fully resolved. Seven hours after the administration of the suppository, the severity of pain increased and the patient took a second vaginal suppository (50 mg CBD:50 mg CBG:250 mg PEA). Once again, the pain was reduced considerably (VAS 1), but did not disappear entirely. Symptoms of severe pain continued to reoccur after 10 hours and the patient took the last dose (50 mg CBD:50 mg CBG:250 mg PEA), and the pain was entirely resolved and there was no need for further medications.

At her next menstrual period, the same patient was instructed to take the cannabinoid vaginal suppository (25 mg CBD:25 mg CBG:250 mg PEA) one day before the start of the bleeding when the muscle cramps started. At the first onset of the cramps, the patient took a vaginal suppository (25 mg CBD:25 mg CBG:250 mg PEA). The patient reported that the cramps were completely relieved within 20 minutes and did not reappear for 10 hours. The regimen was repeated after 10 hours upon emergence of severe pain and cramps. Pain and cramps subsided to mild levels upon vaginal suppository administration and the patient reported that the cramps and pain did not re-occur until the following day when bleeding started. The patient reported severe cramps, lower back and abdominal pain (VAS 6/10) at day one of her subsequent menstrual period. After enduring the pain for 60 minutes and recording that the severity of pain and cramps was increasing (VAS 7/10) the patient inserted the above-mentioned vaginal suppository (25 mg CBD:25 mg CBG:250 mg PEA). Within 30 minutes, the pain began to subside and by 45 minutes was lessened to VAS 3.5 and the cramps were reduced substantially. 6 hours after the administration of the suppository, the severity of pain increased and the patient took a second vaginal suppository (25 mg CBD:25 mg CBG:250 mg PEA). Once again, the pain was reduced considerably (VAS 3). Symptoms of severe pain continued to reoccur after 6-8 hours for the next 18 hours and each time the cramps and pain was substantially reduced by a 25 mg CBD:25 mg CBG:250 mg PEA vaginal suppository.

### Example 2—Synergistic Pain Relief of Menstrual Pain Using Combination of CBD, CBG and PEA Delivered Using a Vaginal Suppository

In a separate trial, the same patient from Example 1 (a female patient, aged 36, with a history of severe menstrual pain) was administered formulations containing CBD only, CBG only, PEA only and a CBD+CBG+PEA combination, each delivered using vaginal suppositories, to treat menstrual pain during another menstrual cycle and menstrual period (i.e. a separate menstrual cycle and menstrual period than in Example 1). Specifically, the formulations were individually/separately administered, with each single formulation being administered on a separate day of the patient's menstrual period. The table below provides a comparison of visual analogue pain scores (VAS scores) in relation to menstrual pain reported by the patient for the CBD only, CBG only, PEA only and CBD+CBG+PEA combination treatments. The effectiveness of the CBD+CBG+PEA combination in comparison to the compounds alone, as reflected in VAS score comparison, is clearly seen. The combination of CBD+CBG+PEA leads to a higher level of pain relief and a longer duration of pain relief compared to the additive effects and the duration of the effects for each compound individually. This enhanced pain relief effect over each single compound is unexpected and offers advantages in pain relief management to the user. This profile shows that the ratios of the compounds used in the treatment (i.e. ratios between about 25:25:250 of CBG:CBG:PEA to 50:50:250 of CBG:CBG:PEA, and preferably about 50:50:250 of CBG:CBG:PEA) results in a synergistic pain relief effect.

Comparison of visual analogue pain scores (VAS scores) for CBD only, CBG only, PEA only and CBD+CBG+PEA combinations delivered using vaginal suppositories.

| | | Onset of | | | Magnitude of pain |
|---|---|---|---|---|---|
| | Baseline | effect | VAS at | Duration of | reduction from |
| | VAS | (minutes) | max effect | effect (hours) | baseline |
| CBD 50 mg | 7 | 20 | 5 | 5 hours | 2 |
| CBG 50 mg | 8 | NA | 8 | NA | 0 |
| PEA 250 mg | 7 | 30 | 5 | 6 hours | 2 |
| CBD 50 mg + | 7.5 | 20 | 1 | 8 hours | 6.5 |
| CBG 50 mg + | | | | | |
| PEA 250 mg | | | | | |

### Example 3—Rectal Suppository for Targeted Treatment of Symptoms Related to Hemorrhoidal Pain

A 70 year old male patient with painful hemorrhoids was instructed to take a rectal suppository containing 50 mg CBDA: 50 mg CBG:250 mg PEA. This dose was to be repeated every 8 hours. No other medication was to be taken. The patient reported significant pain relief and reduction of itchiness 15 minutes after rectal administration of the suppository. The pain levels remained manageable for 12 hours.

### Example 4—Use in Conjunction with TENS Machine

A female patient, aged 36, with a history of severe lower back pain and period cramps was advised to administer cannabinoid infused body oil (100 mg CBD:100 mg CBG:25 mg PEA:30 mg Camphor:60 mg Menthol:1740 mg grapeseed oil) to the skin and to use a transcutaneous electrical nerve stimulation (TENS) machine for 1 hour. Patient reported significant pain and cramp reduction.

### Example 5—Use in Conjunction with a Vaginal Dilator

A female cervical cancer survivor, aged 42, who had cervical surgery and radiation therapy, used to use a vaginal dilator at least twice a week for a total of 10 minutes per session to treat vaginal stenosis. The patient reported significant pain, vaginal spasms and discomfort during these rehabilitation sessions. The patient was instructed to take a vaginal suppository containing 50 mg CBD:50 mg CBG:250 mg PEA 30 minutes prior to insertion of the vaginal dilator. The patient reported good levels of lubrication while inserting the vaginal dilator. The patient reported significant muscle relaxation and pain reduction during the insertion of the vaginal dilator.

### Example 6—Use in Conjunction with Massage Gun

A male patient, aged 42, with severe neck and upper back pain, muscle spasms and tension headaches was instructed to administer cannabinoid infused body oil (100 mg CBD:100 mg CBG:25 mg PEA:30 mg Capsaicin:60 mg Menthol:1740 mg MCT oil) to the skin and massage the area. A portable massage gun was used in combination with the body oil. The patient reported warming up in the area, and significant reduction in muscle spasms and neck pain. The muscle tension reoccurred the next day (within 18 hours) and the body oil was administered again, which substantially reduced the pain.

### Example 7—Use in Conjunction with Heat Pad

A male patient, aged 42, with severe neck and upper back pain and muscle spasms was instructed to administer cannabinoid infused body oil (100 mg CBD:100 mg CBG:25 mg PEA:30 mg Camphor:60 mg Menthol:1740 mg grapeseed oil) to the skin and to heat up the area using a heat pad. The patient reported considerable muscle relaxation and reduction of neck pain 20 minutes after administration of the body oil.

### Example 8—Treatment of Pain Associated with Pelvic Pain

A 43 year old female patient with chronic pelvic pain was advised to take a capsule in capsule dosage where the inner capsule contained 200 mg PEA, 50 mg CBDA and the outer capsule contained pinene (31%, 24.8 mg α-pinene, 6.2 mg β-pinene), camphene (15%, 15.0 mg), anethol (4%, 4.0 mg), borneol (10%, 10.0 mg), cineol (3%, 3.0 mg), fenchone (4%, 4.0 mg), and resveratrol (10%, 10 mg) in olive oil three time per day for 30 days. The patient reported a significant reduction in pain levels, improved sleep quality and improved quality of life. The patient's level of painkiller use was decreased compared to the month prior to initiation of this treatment.

### Example 9—Treatment of Pain Associated with Pelvic Pain

A 72 year old male patient with prostatitis and chronic pelvic pain was advised to take a capsule in capsule dosage where the inner capsule contained 200 mg PEA:50 mg CBD:50 mg CBG and the outer capsule contained pinene (31%, 24.8 mg α-pinene, 6.2 mg β-pinene), camphene (15%, 15.0 mg), anethol (4%, 4.0 mg), borneol (10%, 10.0 mg), cineol (3%, 3.0 mg), and fenchone (4%, 4.0 mg) in olive oil three times per day for 30 days. The patient reported a significant reduction in pain levels, improved sleep quality and improved quality of life. The patient's level of painkiller use was decreased compared to the month prior to initiation of this treatment.

### Example 10—Treatment of Pain Related to Migraines

A 33 year old female patient with migraine and cluster headaches (VAS 6/10, at least 5 headache days a month) was advised to take each of the following formulations individually, with each single formulation being taken for at least 1 month and delivered sublingually:

$$1. 0.5 \% Peppermint essential oil + 0.5 \% Lavender essential oil +$$

$$CBD 100 mg + CBG 100 mg in Jujuba oil + 50 mg PEA$$

$$2. 0.5 \% Chamomile essential oil + 0.5 \% Rosemary essential oil + CBD$$

$$100 mg + CBG 50 mg in Jujuba oil + 50 mg PEA$$

$$3. 0.25 \% Lavender essential oil + 0.25 \% Peppermint essential oil +$$

$$0.25 \% Lemongrass essential oil + CBD 50 mg + CBG 100 mg +$$

$$100 mg PEA in MCT oil$$

$$4. 0.5 \% Frankincense essential oil + 0.5 \% Copaiba essential oil +$$

$$100 mg CBD + 100 mg CBG + 100 mg PEA$$

Accordingly, the patient was treated with each of the above four formulations individually via sublingual delivery, each for at least 1 month. The patient reported improvement in the severity and frequency of symptoms using all of the above-noted formulations for at least 1 month. Specifically, for each of the four formulations, the patient reported a significant reduction in the frequency of headache days, intensity of migraine attacks, and the amount of painkillers taken. Also, for each formulation, the patient reported significant reduction in anxiety, improved sleep and improved mood.

### Example 11—Treatment of Pain Related to Sports Trauma

A 40 year old male patient with muscle fatigue and cramps after heavy exercise was advised to massage his calves for 30 minutes with each of the following formulations individually, with each single formulation being administered to the calves after separate sessions of heavy exercise:

$$1. 0.5 \% lavender + 0.5 \% rosemary + 0.25 \% marjoram + 50 mg PEA +$$

$$100 mg CBD in grapeseed oil$$

$$2. 0.5 \% geranium + 0.5 \% rosemary + 0.25 \% marjoram + 50 mg PEA +$$

$$100 mg CBD in grapeseed oil$$

$$3. 1 \% Birch Essential oil + 0.5 \% Peppermint oil + 0.5 \% Eucalyptus$$

$$Essential Oil + 50 mg PEA + 100 mg CBD in grapeseed oil$$

Accordingly, the patient was treated with each of the above formulations individually, each after separate sessions of heavy exercise. The patient reported improvement in the muscle cramps and muscle pain using all of the above-noted formulations. Specifically, for each of the three formulations, the patient reported a significant reduction in muscle cramps and muscle pain.

### Example 12—Treatment of Pain Related to Sexual Intercourse

A post-menopausal female patient, age 60, with a history of vaginal dryness and dyspareunia (painful intercourse), was instructed to use a vaginal suppository (25 mg CBD:25 mg CBG:100 mg PEA:200 mg resveratrol) 20 minutes prior to intercourse. The patient reported improved lubrication, muscle relaxation and lack of pain during the intercourse.

### Example 13—Treatment of Insomnia and Improvement of Sleep Quality

A female subject, age 36, who has acute insomnia due to anxiety, was instructed to take sublingually 100 mg CBD:100 mg CBG oil:25 mg PEA:45 mg menthol:755 mg grapeseed oil. The patient was instructed to keep the oil under her tongue for 5 minutes. She reported that prior to taking the solution she had difficulty falling asleep and had poor sleep quality (waking up frequently, problems going back to sleep after waking up, anxiety, and brain racing). She reported that starting with her first dose before bedtime, she experienced a drastic (80-90%) improvement in the quality of her sleep (decline in insomnia and symptoms). She fell asleep faster and had fewer/no sleep interruptions. Subject is reported to be more relaxed, and well rested.

### INTERPRETATION OF TERMS

Unless the context clearly requires otherwise, throughout the description and the claims:
“comprise”, “comprising”, and the like are to be construed in an inclusive sense, as opposed to an exclusive or exhaustive sense; that is to say, in the sense of “including, but not limited to”;
“connected”, “coupled”, or any variant thereof, means any connection or coupling, either direct or indirect, between two or more elements; the coupling or connection between the elements can be physical, logical, or a combination thereof;
“herein”, “above”, “below”, and words of similar import, when used to describe this specification, shall refer to this specification as a whole, and not to any particular portions of this specification;
“or”, in reference to a list of two or more items, covers all of the following interpretations of the word: any of the items in the list, all of the items in the list, and any combination of the items in the list;
the singular forms “a”, “an”, and “the” also include the meaning of any appropriate plural forms.

Words that indicate directions such as “vertical”, “transverse”, “horizontal”, “upward”, “downward”, “forward”, “backward”, “inward”, “outward”, “left”, “right”, “front”, “back”, “top”, “bottom”, “below”, “above”, “under”, and the like, used in this description and any accompanying claims (where present), depend on the specific orientation of the apparatus described and illustrated. The subject matter described herein may assume various alternative orientations. Accordingly, these directional terms are not strictly defined and should not be interpreted narrowly.

For example, while processes or blocks are presented in a given order, alternative examples may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed in parallel, or may be performed at different times.

In addition, while elements are at times shown as being performed sequentially, they may instead be performed simultaneously or in different sequences. It is therefore intended that the following claims are interpreted to include all such variations as are within their intended scope.

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

Various features are described herein as being present in “some embodiments”. Such features are not mandatory and may not be present in all embodiments. Embodiments of the invention may include zero, any one or any combination of two or more of such features. This is limited only to the extent that certain ones of such features are incompatible with other ones of such features in the sense that it would be impossible for a person of ordinary skill in the art to construct a practical embodiment that combines such incompatible features. Consequently, the description that “some embodiments” possess feature A and “some embodiments” possess feature B should be interpreted as an express indication that the inventors also contemplate embodiments which combine features A and B (unless the description states otherwise or features A and B are fundamentally incompatible).

It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples, but should be given the broadest interpretation consistent with the description as a whole. While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are consistent with the broadest interpretation of the specification as a whole.

### REFERENCES

All references referred to herein are incorporated by reference in their entireties for all purposes.
1. Raja, S. N., Carr, D. B., Cohen, M., Finnerup, N. B., Flor, H., Gibson, S., Keefe, F. J., Mogil, J. S., Ringkamp, M., Sluka, K. A., Song, X. J., Stevens, B., Sullivan, M. D., Tutelman, P. R., Ushida, T., & Vader, K. (2020). The revised International Association for the Study of Pain definition of pain: concepts, challenges, and compromises. Pain, 161 (9), 1976-1982. https://doi.org/10.1097/j.pain.0000000000001939.
2 Nicholson B. (2006). Differential diagnosis: nociceptive and neuropathic pain. The American journal of managed care, 12 (9 Suppl), S256-S262.
3. Merskey, H. (Ed.). (1986). Classification of chronic pain: Descriptions of chronic pain syndromes and definitions of pain terms. Pain, Suppl 3, 226.
4. Treede, R. D., Rief, W., Barke, A., Aziz, Q., Bennett, M. I., Benoliel, R., Cohen, M., Evers, S., Finnerup, N. B., First, M. B., Giamberardino, M. A., Kaasa, S., Kosek, E., Lavand'homme, P., Nicholas, M., Perrot, S., Scholz, J., Schug, S., Smith, B. H., Svensson, P., . . . . Wang, S. J. (2015). A classification of chronic pain for ICD-11. Pain, 156 (6), 1003-1007. https://doi.org/10.1097/j.pain.0000000000000160.
5. Breivik, H., Collett, B., Ventafridda, V., Cohen, R., & Gallacher, D. (2006). Survey of chronic pain in Europe: prevalence, impact on daily life, and treatment. European journal of pain (London, England), 10 (4), 287-333. https://doi.org/10.1016/j.ejpain.2005.06.009.
6. Goldberg, D. S., & McGee, S. J. (2011). Pain as a global public health priority. BMC public health, 11, 770. https://doi.org/10.1186/1471-2458-11-770
7. Latthe, P., Latthe, M., Say, L. et al. (2006). WHO systematic review of prevalence of chronic pelvic pain: a neglected reproductive health morbidity. BMC Public Health 6, 177. https://doi.org/10.1186/1471-2458-6-177.
8. Zondervan, K. T., Yudkin, P. L., Vessey, M. P., Dawes, M. G., Barlow, D. H., & Kennedy, S. H. (1998). The prevalence of chronic pelvic pain in women in the United Kingdom: a systematic review. British journal of obstetrics and gynaecology, 105 (1), 93-99. https://doi.org/10.1111/j. 1471-0528.1998.tb09357.x.
9. Grace, V. M., & Zondervan, K. T. (2004). Chronic pelvic pain in New Zealand: prevalence, pain severity, diagnoses and use of the health services. Australian and New Zealand journal of public health, 28 (4), 369-375. https://doi.org/10.1111/j.1467-842x.2004.tb00446.x.
10. Daniels J P, Khan K S. (2010) Chronic pelvic pain in women. BMJ. 341: c4834 https://doi.org/10.1136/bmj.c4834.
11. Ju, H., Jones, M., & Mishra, G. (2014). The prevalence and risk factors of dysmenorrhea. Epidemiologic reviews, 36, 104-113. https://doi.org/10.1093/epirev/mxt009.
12. Armour M, Ferfolja T, Curry C, Hyman M S, Parry K, Chalmers K J, Smith C A, MacMillan F, Holmes K. The Prevalence and Educational Impact of Pelvic and Menstrual Pain in Australia: A National Online Survey of 4202 Young Women Aged 13-25 Years. J Pediatr Adolesc Gynecol. 2020 October; 33 (5): 511-518. doi: 10.1016/j.jpag.2020.06.007. Epub 2020 Jun. 13. PMID: 32544516.
13. “Dysmenorrhea: Painful Periods”. https://www.acog.org/womens health/faqs/dysmenorrhea-painful-periods
14. “Dysmenorrhea”. https://www.hopkinsmedicine.org/health/conditions-and diseases/dysmenorrhea
15. Samuel, Essie, et al. (2017) Managing Chronic Pain: A Review of the CDC Guidelines. U S Pharm. 42 (10): HS-31-HS-34. https://www.uspharmacist.com/article/managing-chronic-pain-a-review-of-the-cdc guidelines
16. Gatchel, R. J., McGeary, D. D., McGeary, C. A., & Lippe, B. (2014). Interdisciplinary chronic pain management: Past, present, and future. American Psychologist, 69 (2), 119-130. https://doi.org/10.1037/a0035514.
17. G. Varrassi, G. Müller-Schwefe, J. Pergolizzi, A. Orónska, B. Morlion, P. Mavrocordatos, C. Margarit, C. Mangas, W. Jaksch, F. Huygen, B. Collett, M. Berti, D. Aldington & K. Ahlbeck (2010) Pharmacological treatment of chronic pain the need for CHANGE, Current Medical Research and Opinion, 26:5, 1231-1245, https://doi.org/10.1185/03007991003689175.
18. French L. (2005). Dysmenorrhea. American family physician, 71 (2), 285-291.
19. Zeev Harel (2012) Dysmenorrhea in adolescents and young adults: an update on pharmacological treatments and management strategies, Expert Opinion on Pharmacotherapy, 13:15, 2157-2170. https://doi.org/10.1517/14656566.2012.725045.
20. Zeev Harel (2008) Dysmenorrhea in adolescents and young adults: from pathophysiology to pharmacological treatments and management strategies, Expert Opinion on Pharmacotherapy, 9:15, 2661-2672. https://doi.org/10.1517/14656566.9.15.2661.
21. Chen X. Chen, Janet S. Carpenter, Michelle LaPradd, Susan Ofner, and J. Dennis Fortenberry. Journal of Women's Health. September 2021.1334-1343. http://doi.org/10.1089/jwh.2020.8581.
22. B. Halliwell, R. Aeschbach, J. Loliger, O. I. Aruoma, The characterization of antioxidants, Food and Chemical Toxicology, Volume 33, Issue 7, 1995, Pages 601-617, ISSN 0278-6915,
23. Understanding diversity in oxidative status and oxidative stress: the opportunities and challenges ahead, David Costantini, Author and article information J Exp Biol (2019) 222 (13): jeb194688., https://doi.org/10.1242/jeb. 194688
24. Scott K. Powers, Rafael Deminice, Mustafa Ozdemir, Toshinori Yoshihara, Matthew P. Bomkamp, Hayden Hyatt, Exercise-induced oxidative stress: Friend or foe?, Journal of Sport and Health Science, Volume 9, Issue 5, 2020, Pages 415-425, ISSN 2095-2546, https://doi.org/10.1016/j.jshs.2020.04.001.
25. Peoples, J. N., Saraf, A., Ghazal, N. et al. Mitochondrial dysfunction and oxidative stress in heart disease. Exp Mol Med 51, 1-13 (2019). https://doi.org/10.1038/s12276-019-0355-7
26. Singh, A.; Kukreti, R.; Saso, L.; Kukreti, S. Oxidative Stress: A Key Modulator in Neurodegenerative Diseases. Molecules 2019, 24, 1583. https://doi.org/10.3390/molecules24081583
27. Liguori I, Russo G, Curcio F, Bulli G, Aran L, Della-Morte D, Gargiulo G, Testa G, Cacciatore F, Bonaduce D, Abete P. Oxidative stress, aging, and diseases. Clin Interv Aging. 2018 Apr. 26; 13:757-772. doi: 10.2147/CIA.S158513. PMID: 29731617; PMCID: PMC5927356.
28. Zhu-Min Wang, Yong-Cai Chen, Da-Peng Wang, Resveratrol, a natural antioxidant, protects monosodium iodoacetate-induced osteoarthritic pain in rats, Biomedicine & Pharmacotherapy, Volume 83, 2016, Pages 763-770, ISSN 0753-3322, https://doi.org/10.1016/j.biopha.2016.06.050.
29. Daniel Mendes da Silva, Luiza Azevedo Gross, Ernesto de Paula Guedes Neto, Bruce A. Lessey, Ricardo Francalacci Savaris, The Use of Resveratrol as an Adjuvant Treatment of Pain in Endometriosis: A Randomized Clinical Trial, Journal of the Endocrine Society, Volume 1, Issue 4, 1 Apr. 2017, Pages 359-369, https://doi.org/10.1210/js.2017-00053
30. Di Paola R, Fusco R, Gugliandolo E, Crupi R, Evangelista M, Granese R, Cuzzocrea S. Co-micronized Palmitoylethanolamide/Polydatin Treatment Causes Endometriotic Lesion Regression in a Rodent Model of Surgically Induced Endometriosis. Front Pharmacol. 2016 Oct. 14; 7:382. doi: 10.3389/fphar.2016.00382. PMID: 27790149; PMCID: PMC5063853.
31. Ricciotti E, FitzGerald G A. Prostaglandins and inflammation. Arterioscler Thromb Vasc Biol. 2011 May; 31 (5): 986-1000. doi: 10.1161/ATVBAHA. 110.207449. PMID:
   21508345; PMCID: PMC3081099.
32. Battista N, Di Tommaso M, Bari M and Maccarrone M (2012) The endocannabinoid system: an overview. Front. Behav. Neurosci. 6:9. https://doi.org/10.3389/fnbeh.2012.00009.
33. Zou, S., & Kumar, U. (2018). Cannabinoid Receptors and the Endocannabinoid System: Signaling and Function in the Central Nervous System. International journal of molecular sciences, 19 (3), 833. https://doi.org/10.3390/ijms19030833.
34. Marcu, Jahan P. (2016). Chapter 62—An Overview of Major and Minor Phytocannabinoids (V. R. Preedy, Ed.), Neuropathology of Drug Addictions and Substance Misuse, Vol. #1: Foundations of Understanding, Tobacco, Alcohol, Cannabinoids and Opioids (pp. 672-678). Academic Press. https://doi.org/10.1016/B978-O-12-800213-1.00062-6.
35. Blaszszak-Boxe, Agata. (2014 Oct. 17). Marijuana's History: How One Plant Spread Through the World. LIVESCIENCE. https://www.livescience.com/48337-marijuana-history-how-cannabis-travelled-world.html.
36. Hill, K. P., Palastro, M. D., Johnson, B., & Ditre, J. W. (2017). Cannabis and Pain: A Clinical Review. Cannabis and cannabinoid research, 2 (1), 96-104. https://doi.org/10.1089/can.2017.0017.
37. Pantoja-Ruiz, C. et al. (2022) Cannabis and pain: a scoping review. Brazilian Journal of Anesthesiology (English Edition). 72 (1), 142-151. https://doi.org/10.1016/j.bjane.2021.06.018.
38. Uma Anand et al., (2011) Cannabis and pain: a scoping review. Brazilian Journal of Anesthesiology. Pain Management. 11:4, 395-403 https://www.futuremedicine.com/doi/10.2217/pmt-2020-0110
39. Busse J W, Vankrunkelsven P, Zeng L, Heen A F, Merglen A, Campbell F et al. (2021) Medical cannabis or cannabinoids for chronic pain: a clinical practice guideline. BMJ. 374: n2040. https://doi.org/10.1136/bmj.n2040.
40. Blake, A., Wan, B. A., Malek, L., DeAngelis, C., Diaz, P., Lao, N., Chow, E., & O'Hearn, S. (2017). A selective review of medical cannabis in cancer pain management. Annals of palliative medicine, 6 (Suppl 2), S215-S222. https://doi.org/10.21037/apm.2017.08.05.
41. Xu, D. H., Cullen, B. D., Tang, M., & Fang, Y. (2020). The Effectiveness of Topical Cannabidiol Oil in Symptomatic Relief of Peripheral Neuropathy of the Lower Extremities. Current pharmaceutical biotechnology, 21 (5), 390-402. https://doi.org/10.2174/1389201020666191202111534.
42. Serpell, M., Ratcliffe, S., Hovorka, J., Schofield, M., Taylor, L., Lauder, H. and Ehler, E. (2014), Efficacy of THC/CBD spray in peripheral neuropathic pain. EJP, 18:999-1012. https://doi.org/10.1002/j. 1532-2149.2013.00445.x
43. Geoffrey Guy et al. (2011). Use of cannabidiol/tetrahydrocannabinol compounds in the treatment of neuropathic pain (UK Patent No. GB2439393B). United Kingdom Intellectual Property Office. https://patents.google.com/patent/GB2439393A/en?oq=GB2439393
44. Geoffrey Guy et al. (2007). A combination of cannabinoids for the treatment of peripheral neurophatic pain (PCT Publication WO2007052013A1). World Intellectual Property Organization. https://patents.google.com/patent/WO2007052013Δ1/en?oq=PCT % 2fGB2006% 2f 00 4063+
45. Marinelli L, Balestrino M, Mori L, et al. A randomised controlled cross-over double blind pilot study protocol on THC:CBD oromucosal spray efficacy as an add-on therapy for post-stroke spasticity. BMJ Open. 2017; 7 (9): e016843. Published 2017 Sep. 7. doi: 10.1136/bmjopen-2017-016843
46. Langford, R. M., Mares, J., Novotna, A. et al. A double-blind, randomized, placebo controlled, parallel-group study of THC/CBD oromucosal spray in combination with the existing treatment regimen, in the relief of central neuropathic pain in patients with multiple sclerosis. J Neurol 260, 984-997 (2013). https://doi.org/10.1007/s00415-012-6739-4
47. David J. Rog, Turo J. Nurmikko, Carolyn A. Young. Oromucosal Δ9-tetrahydrocannabinol/cannabidiol for neuropathic pain associated with multiple sclerosis: An uncontrolled, open-label, 2-year extension trial, Clinical Therapeutics. Vol 29, Issue 9, 2007, page 2068-2079. https://doi.org/10.1016/j.clinthera.2007.09.013.
48. Turo J. Nurmikko, Mick G. Serpell, Barbara Hoggart, Peter J. Toomey, Bart J. Morlion, Derek Haines. Sativex successfully treats neuropathic pain characterised by allodynia: A randomised, double-blind, placebo-controlled clinical trial, PAIN®, Volume 133, Issues 1-3, 2007, Pages 210-220. https://doi.org/10.1016/j.pain.2007.08.028.
49. Jeremy R. Johnson, Mary Burnell-Nugent, Dominique Lossignol, Elena Doina Ganae-Motan, Richard Potts, Marie T. Fallon. Multicenter, Double-Blind, Randomized, Placebo-Controlled, Parallel-Group Study of the Efficacy, Safety, and Tolerability of THC:CBD Extract and THC Extract in Patients with Intractable Cancer Related Pain. Journal of Pain and Symptom Management, Volume 39, Issue 2, 2010, Pages 167-179. https://doi.org/10.1016/j.jpainsymman.2009.06.008.
50. Novotna, A., Mares, J., Ratcliffe, S., Novakova, I., Vachova, M., Zapletalova, O., Gasperini, C., Pozzilli, C., Cefaro, L., Comi, G., Rossi, P., Ambler, Z., Stelmasiak, Z., Erdmann, A., Montalban, X., Klimek, A., Davies, P. and (2011), A randomized, double-blind, placebo-controlled, parallel-group, enriched-design study of nabiximols (Sativex®), as add-on therapy, in subjects with refractory spasticity caused by multiple sclerosis. European Journal of Neurology, 18:1122-1131. https://doi.org/10.1111/j. 1468-1331.2010.03328.x.
51. Cox-Georgian, D., Ramadoss, N., Dona, C., & Basu, C. (2019). Therapeutic and Medicinal Uses of Terpenes. Medicinal Plants: From Farm to Pharmacy, 333-359. https://doi.org/10.1007/978-3-030-31269-5_15.
52. Aldred, E. M. (2009). Pharmacology: A handbook for complementary healthcare professionals. Edinburgh: Churchill Livingstone/Elsevier.
53. Russo E. B. (2011). Taming THC: potential cannabis synergy and phytocannabinoid-terpenoid entourage effects. British journal of pharmacology, 163 (7), 1344-1364. https://doi.org/10.1111/j.1476-5381.2011.01238.x
54. Lee, C. B., Ha, U. S., Lee, S. J., Kim, S. W., & Cho, Y. H. (2006). Preliminary experience with a terpene mixture versus ibuprofen for treatment of category III chronic prostatitis/chronic pelvic pain syndrome. World journal of urology, 24 (1), 55-60. https://doi.org/10.1007/s00345-005-0039-x.
55. Guimarães, A. G., Serafini, M. R., & Quintans-Júnior, L. J. (2014). Terpenes and derivatives as a new perspective for pain treatment: a patent review. Expert opinion on therapeutic patents, 24 (3), 243-265. https://doi.org/10.1517/13543776.2014.870154.
56. Erika Liktor-Busa, Attila Keresztes, Justin LaVigne, John M. Streicher and Tally M. Largent-Milnes. Pharmacological Reviews October 2021, 73 (4) 1269-1297; DOI:
   https://doi.org/10.1124/pharmrev.120.000046.
57. Passavanti, M. B., Fiore, M., Sansone, P. et al. (2017). The beneficial use of ultramicronized palmitoylethanolamide as add-on therapy to Tapentadol in the treatment of low back pain: a pilot study comparing prospective and retrospective observational arms. BMC Anesthesiol 17, 171. https://doi.org/10.1186/s12871-017-0461-9.
58. Germini, F., Coerezza, A., Andreinetti, L. et al. (2017), N-of-1 Randomized Trials of Ultra-Micronized Palmitoylethanolamide in Older Patients with Chronic Pain. Drugs Aging 34, 941-952. https://doi.org/10.1007/s40266-017-0506-2.
59. Chirchiglia, D. et al. (2018) Nonsurgical lumbar radiculopathies treated with ultramicronized palmitoylethanolamide (umPEA): A series of 100 cases. Neurol Neurochir Pol 52 (1), 44-47. https://doi.org/10.1016/j.pjnns.2017.11.002.
60. Giammusso, B., Di Mauro, R., & Bernardini, R. (2017). The efficacy of an association of palmitoylethanolamide and alpha-lipoic acid in patients with chronic prostatitis/chronic pelvic pain syndrome: A randomized clinical trial. Archivio Italiano Di Urologia E Andrologia, 89 (1), 17-21. https://doi.org/10.4081/aiua.2017.1.17.
61. Redonyl Ultra 200 mg (Canada). https://www.drugs.com/vet/redonyl-ultra-200-mg can.html. https://www.dechra-us.com/Files/Files/SupportMaterialDownloads/us/US 089-RED.pdf
62. Gabrielsson L, Mattsson S, Fowler C J. Palmitoylethanolamide for the treatment of pain: pharmacokinetics, safety and efficacy. Br J Clin Pharmacol. 2016; 82 (4): 932-942. doi: 10.1111/bcp.13020.
63. Petrosino S, Cordaro M, Verde R, Schiano Moriello A, Marcolongo G, Schievano C, Siracusa R, Piscitelli F, Peritore A F, Crupi R, Impellizzeri D, Esposito E, Cuzzocrea S, Di Marzo V. Oral Ultramicronized Palmitoylethanolamide: Plasma and Tissue Levels and Spinal Anti-hyperalgesic Effect. Front Pharmacol. 2018 Mar. 20; 9:249. doi: 10.3389/fphar.2018.00249.
64. Kriek, Rutger Branded name for ultramicronized palmitoylethanolamide and flawed science, PAIN: March 2016—Volume 157—Issue 3—p 769. doi: 10.1097/j.pain.0000000000000399
65. Bouaziz J, Bar On A, Seidman D S, Soriano D. The Clinical Significance of Endocannabinoids in Endometriosis Pain Management. Cannabis Cannabinoid Res. 2017; 2 (1): 72-80. Published 2017 Apr. 1. doi: 10.1089/can.2016.0035
66. Tartaglia, E., Armentano, M., Giugliano, B., Sena, T., Giuliano, P., Loffredo, C., & Mastrantonio, P. (2015). Effectiveness of the Association N-Palmitoylethanolamine and Transpolydatin in the Treatment of Primary Dysmenorrhea. Journal of pediatric and adolescent gynecology, 28 (6), 447-450. https://doi.org/10.1016/j.jpag.2014.12.011
67. Caruso S, Iraci Sareri M, Casella E, et al. Chronic pelvic pain, quality of life and sexual health of women treated with palmitoylethanolamide and α-lipoic acid. Minerva Ginecologica. 2015 October; 67 (5): 413-419. PMID: 26491823.
68. Lo Monte G, Soave I, Marci R. [Administration of micronized palmitoylethanolamide (PEA)-transpolydatin in the treatment of chronic pelvic pain in women affected by endometriosis: preliminary results]. Minerva Ginecologica. 2013 August; 65 (4): 453-463. PMID: 24051945.
69. Emilio Giugliano, Elisa Cagnazzo, Ilaria Soave, Giuseppe Lo Monte, Jean Marie Wenger, Roberto Marci. The adjuvant use of N-palmitoylethanolamine and transpolydatin in the treatment of endometriotic pain. European Journal of Obstetrics & Gynecology and Reproductive Biology, Volume 168, Issue 2, 2013, Pages 209-213, https://doi.org/10.1016/j.ejogrb.2013.01.009.
70. Nahin R. L., Barnes P. M., Stussman B. J., Bloom B. Costs of Complementary and Alternative Medicine (Cam) and Frequency of Visits to Cam Practitioners: United States, 2007. Natl. Health Stat. Rep. 2009; 18:1-14.
71. Complementary and alternative approaches to the treatment of persistent musculoskeletal pain. Weiner D K, Ernst E. Clin J Pain. 2004 July-August; 20 (4): 244-55.
72. Hexaresearch. Global Herbal Medicine Market Size, Value, 2014-2024. Hexaresearch; Pune, M H, India: 2017.
73. Use of complementary therapies for arthritis among patients of rheumatologists. Rao J K, Mihaliak K, Kroenke K, Bradley J, Tierney W M, Weinberger M Ann Intern Med. 1999 Sep. 21; 131 (6): 409-16.
74. Fautrel B, Adam V, St-Pierre Y, Joseph L, Clarke A E, Penrod J R. Use of complementary and alternative therapies by patients self-reporting arthritis or rheumatism: results from a nationwide Canadian survey. J Rheumatol. 2002 November; 29 (11): 2435-41. PMID: 12415605.
75. Rosenberg E I, Genao I, Chen I, Mechaber A J, Wood J A, Faselis C J, Kurz J, Menon M, O'Rorke J, Panda M, Pasanen M, Staton L, Calleson D, Cykert S. Complementary and alternative medicine use by primary care patients with chronic pain. Pain Med. 2008 November; 9 (8): 1065-72. doi: 10.1111/j. 1526-4637.2008.00477.x. Epub 2008 Jun. 18. PMID: 18564996.
76. Cheng B, Liu Y, Tian J, Gao R, Liu Y. Complementary and Alternative Medicine for the Treatment of Insomnia: An Overview of Scientific Evidence from 2008 to 2018. Curr Vasc Pharmacol. 2020; 18 (4): 307-321.
   doi: 10.2174/1570161117666190506111239. PMID: 31057109.
77. Ohayo M M. Severe Hot Flashes Are Associated With Chronic Insomnia. Arch Intern Med. 2006; 166 (12): 1262-1268. doi: 10.1001/archinte.166.12.1262
78. Jobling Phillip, O'Hara Kate, Hua Susan. (2014). Female reproductive tract pain: targets, challenges, and outcomes. Frontiers in Pharmacology 5. https://doi.org/10.3389/fphar.2014.00017
79. Maddern Jessica, Grundy Luke, Castro Joel, Brierley Stuart M. (2020) Pain in Endometriosis. Frontiers in Cellular Neuroscience 14. https://doi.org/10.3389/fncel.2020.590823.
80. Ran Guo, Li-Hua Chen, Chungen Xing, Tong Liu. Pain regulation by gut microbiota: molecular mechanisms and therapeutic potential. British Journal of Anaesthesia. Volume 123, Issue 5, 2019, Pages 637-654, ISSN 0007-0912, https://doi.org/10.1016/j.bja.2019.07.026. (https://www.sciencedirect.com/science/article/pii/S0007091219306385)
81. Ghasemian M, Owlia S, Owlia M B. Review of Anti-Inflammatory Herbal Medicines. Adv Pharmacol Sci. 2016; 2016:9130979. doi: 10.1155/2016/9130979.
82. Jahromi, Pirvulescu, I., Candido, K. D., & Knezevic, N. N. (2021). Herbal Medicine for Pain Management: Efficacy and Drug Interactions. Pharmaceutics, 13 (2), 251. https://doi.org/10.3390/pharmaceutics13020251.
83. Phytochemicals of AzadirachtaIndica Source of Active Medicinal Constituent Used for Cure of Various Diseases: A Review. Volume 64, Issue 1, 2020. Journal of Scientific Research Institute of Science, Banaras Hindu University, Varanasi, India.
84. Yatoo M I, Gopalakrishnan A, Saxena A, Parray O R, Tufani N A, Chakraborty S, Tiwari R, Dhama K, Iqbal H M N. Anti-Inflammatory Drugs and Herbs with Special Emphasis on Herbal Medicines for Countering Inflammatory Diseases and Disorders-A Review. Recent Pat Inflamm Allergy Drug Discov. 2018; 12 (1): 39-58. doi: 10.2174/1872213X12666180115153635. PMID: 29336271.
85 Budavari S. The Merck Index: An Encyclopedia of Chemicals, Drugs, and Biologicals. 11th ed. Merck; Rahway, N J, USA: 1989. p. 11.
86. The peri-operative implications of herbal medicines. Hodges P J, Kam P C. Anaesthesia. 2002 September; 57 (9): 889-99.
87. Fetrow C. W., Avila J. R. The Complete Guide to Herbal Medicines. Simon and Schuster; New York, N Y, USA: 2000.
88. Ginger treatment of hyperemesis gravidarum. Fischer-Rasmussen W, Kjaer S K, Dahl C, Asping U Eur J Obstet Gynecol Reprod Biol. 1991 Jan. 4; 38 (1): 19-24.
89. Ginger. Grant K L, Lutz R B Am J Health Syst Pharm. 2000 May 15; 57 (10): 945-7.
90. Gingerols and related analogues inhibit arachidonic acid-induced human platelet serotonin release and aggregation. Koo K L, Ammit A J, Tran V H, Duke C C, Roufogalis B D Thromb Res. 2001 Sep. 1; 103 (5): 387-97.
91. Acute effects of dietary ginger on quadriceps muscle pain during moderate-intensity cycling exercise. Black C D, Oconnor P J Int J Sport Nutr Exerc Metab. 2008 December; 18 (6): 653-64.
92. The use of ginger (Zingiber officinale) for the treatment of pain: a systematic review of clinical trials. Terry R, Posadzki P, Watson L K, Ernst E. Pain Med. 2011 December; 12 (12): 1808-18.
93. Curcumin treatment attenuates pain and enhances functional recovery after incision. Sahbaie P, Sun Y, Liang D Y, Shi X Y, Clark J D Anesth Analg. 2014 June; 118 (6): 1336-44.
94. Curcumin and autoimmune disease. Bright J J Adv Exp Med Biol. 2007; 595 ( ) 425-51.
95. Turmeric supplementation improves the quality of life and hematological parameters in breast cancer patients on paclitaxel chemotherapy: A case series. Kalluru H, Kondaveeti S S, Telapolu S, Kalachaveedu M Complement Ther Clin Pract. 2020 November; 41 ( ) 101247.
96. Capsaicin. Neuropathic pain: playing with fire . . . . Prescrire Int. 2010 August; 19 (108): 153-5.
97. Pharmacokinetic analysis of capsaicin after topical administration of a high concentration capsaicin patch to patients with peripheral neuropathic pain. Babbar S, Marier J F, Mouksassi M S, Beliveau M, Vanhove G F, Chanda S, Bley K Ther Drug Monit. 2009 August; 31 (4): 502-10.
98. The effect of wound instillation of a novel purified capsaicin formulation on postherniotomy pain: a double-blind, randomized, placebo-controlled study. Aasvang E K, Hansen J B, Malmstrøm J, Asmussen T, Gennevois D, Struys M M, Kehlet H Anesth Analg. 2008 July; 107 (1): 282-91.
99. Efficacy and Safety of Diclofenac+Capsaicin Gel in Patients with Acute Back/Neck Pain: A Multicenter Randomized Controlled Study. Predel H G, Ebel-Bitoun C, Peil B, Weiser T W, Lange R Pain Ther. 2020 June; 9 (1): 279-296.
100. Expert Opinion: Exploring the Effectiveness and Tolerability of Capsaicin 179 mg Cutaneous Patch and Pregabalin in the Treatment of Peripheral Neuropathic Pain. Huygen F, Kern K U, Pérez C J Pain Res. 2020; 13 ( ) 2585-2597.
101. Jolayemi A T, Ojewole J A. Comparative anti-inflammatory properties of Capsaicin and ethyl-aAcetate extract of Capsicum frutescens linn [Solanaceae] in rats. Afr Health Sci. 2013 June; 13 (2): 357-61. doi: 10.4314/ahs.v13i2.23. PMID: 24235936; PMCID: PMC3824481.
102. Effect of cream, prepared with Tripterygium wilfordii Hook F and other four medicinals, on joint pain and swelling in patients with rheumatoid arthritis: a double blinded, randomized, placebo controlled clinical trial. Jiao J, Tang X, Gong X, Yin H, Jiang Q, Wei C J Tradit Chin Med. 2019 February; 39 (1): 89-96.
103. A Chinese herb Tripterygium wilfordii Hook F in the treatment of rheumatoid arthritis: mechanism, efficacy, and safety. Bao J, Dai S M. Rheumatol Int. 2011 September; 31 (9): 1123-9.
104. Foods and supplements in the management of migraine headaches. Sun Edelstein C, Mauskop A. Clin J Pain. 2009 June; 25 (5): 446-52.
105. The anti-migraine component of butterbur extracts, isopetasin, desensitizes peptidergic nociceptors by acting on TRPA1 cation channel. Benemei S, De Logu F, Li Puma S, Marone I M, Coppi E, Ugolini F, Liedtke W, Pollastro F, Appendino G, Geppetti P, Materazzi S, Nassini R Br J Pharmacol. 2017 September; 174 (17): 2897-2911.
106. Efficacy and Safety of White Willow Bark (Salix alba) Extracts. Shara M, Stohs S J Phytother Res. 2015 August; 29 (8): 1112-6.
107. Efficacy and safety of willow bark extract in the treatment of osteoarthritis and rheumatoid arthritis: results of 2 randomized double-blind controlled trials. Biegert C, Wagner I, Lüdtke R, Kötter I, Lohmüller C, Günaydin I, Taxis K, Heide L J Rheumatol. 2004 November; 31 (11): 2121-30.
108. Salari S, Amiri M S, Ramezani M, Moghadam A T, Elyasi S, Sahebkar A, Emami S A. Ethnobotany, Phytochemistry, Traditional and Modern Uses of Actaea racemosa L. (Black cohosh): A Review. Adv Exp Med Biol. 2021; 1308:403-449. doi: 10.1007/978-3-030-64872-5_24. PMID: 33861455.
109. Kim Y S, Kim E K, Nawarathna W P A S, Dong X, Shin W B, Park J S, Moon S H, Park P J. Immune-Stimulatory Effects of Althaea rosea Flower Extracts through the MAPK Signaling Pathway in RAW264.7 Cells. Molecules. 2017 Apr. 25; 22 (5): 679. doi: 10.3390/molecules22050679. PMID: 28441343; PMCID: PMC6154003.
110. Ali Esmail Al-Snafi et al/Int. J. PharmTech Res.2013,5 (3). The Pharmaceutical Importance of Althaea officinalis and Althaea rosea: A Review
111. Badoni Semwal R, Semwal D K, Combrinck S, Cartwright-Jones C, Viljoen A. Lawsonia inermis L. (henna): ethnobotanical, phytochemical and pharmacological aspects. J Ethnopharmacol. 2014 Aug. 8; 155 (1): 80-103. doi: 10.1016/j.jep.2014.05.042. Epub 2014 Jun. 2. PMID: 24886774.
112. Rakhshandeh H, Ghorbanzadeh A, Negah S S, Akaberi M, Rashidi R, Forouzanfar F. Pain-relieving effects of Lawsonia inermis on neuropathic pain induced by chronic constriction injury. Metab Brain Dis. 2021 October; 36 (7): 1709-1716. doi: 10.1007/s11011-021-00773-w. Epub 2021 Jun. 25. PMID: 34169409.
113. Rekik D M, Ben Khedir S, Daoud A, Ksouda Moalla K, Rebai T, Sahnoun Z. Wound Healing Effect of Lawsonia inermis. Skin Pharmacol Physiol. 2019; 32 (6): 295-306. doi: 10.1159/000501730. Epub 2019 Aug. 29. PMID:
   31466077.
114. Kim Y J, Lee J Y, Kim H J, et al. Anti-Inflammatory Effects of Angelica sinensis (Oliv.) Diels Water Extract on RAW 264.7 Induced with Lipopolysaccharide. Nutrients. 2018; 10 (5): 647. Published 2018 May 21. doi: 10.3390/nu10050647
115. Chao, W W., Lin, B F. Bioactivities of major constituents isolated from Angelica sinensis (Danggui). Chin Med 6, 29 (2011). https://doi.org/10.1186/1749-8546-6-29 116. Proc. West. Pharmacol. Soc. 45:76-78 (2002).
116. Preliminary Investigation of the Anti inflammatory Properties of an Aqueous Extract from Morinda citrifolia (Noni). MARSHA LYN G. McKOY, EVERTON A. THOMAS & OSWALD R. SIMON
117. E. Dussossoy, P. Brat, E. Bony, F. Boudard, P. Poucheret, C. Mertz, J. Giaimis, A. Michel, Characterization, anti-oxidative and anti-inflammatory effects of Costa Rican noni juice (Morinda citrifolia L.), Journal of Ethnopharmacology, Volume 133, Issue 1, 2011, Pages 108-115, ISSN 0378-8741, https://doi.org/10.1016/j.jep.2010.08.063. (https://www.sciencedirect.com/science/article/pii/S0378874110006501)
118. Toshihiro Akihisa, Kazumi Matsumoto, Harukuni Tokuda, Ken Yasukawa, Ken-ichi Seino, Katsuo Nakamoto, Hideki Kuninaga, Takashi Suzuki, and Yumiko Kimura. (2007). Anti-inflammatory and Potential Cancer Chemopreventive Constituents of the Fruits of Morinda citrifolia (Noni). Journal of Natural Products 70 (5), 754-757. https://doi.org/10.1021/np0680650
119. Mahboubi M. Evening Primrose (Oenothera biennis) Oil in Management of Female Ailments. J Menopausal Med. 2019; 25 (2): 74-82. doi: 10.6118/jmm. 18190
120. Budeiri D, Li Wan Po A, Dornan J C. Is evening primrose oil of value in the treatment of premenstrual syndrome? Control Clin Trials. 1996 February; 17 (1): 60-8. doi: 10.1016/0197-2456 (95) 00082-8. PMID: 8721802.
121. Lampariello L R, Cortelazzo A, Guerranti R, Sticozzi C, Valacchi G. The Magic Velvet Bean of Mucuna pruriens. J Tradit Complement Med. 2012; 2 (4): 331-339. doi: 10.1016/s2225-4110 (16) 30119-5
122. Pathania R, Chawla P, Khan H, Kaushik R, Khan M A. An assessment of potential nutritive and medicinal properties of Mucuna pruriens: a natural food legume. 3 Biotech. 2020 June; 10 (6): 261. doi: 10.1007/s13205-020-02253-x. Epub 2020 May 20. PMID: 32477848; PMCID: PMC7239958.
123. Bian T, Corral P, Wang Y, et al. Kava as a Clinical Nutrient: Promises and Challenges. Nutrients. 2020; 12 (10): 3044. 2020 Oct. 5. doi: 10.3390/nu12103044.
124. Romm, Aviva. (2016). Botanical Medicine for Women's Health. Elsevier Canada. ISBN-13 978-0443072772
125. Wei-ling Pu, Meng-ying Zhang, Ru-yu Bai, Li-kang Sun, Wen-hua Li, Ying-li Yu, Yue Zhang, Lei Song, Zhao-xin Wang, Yan-fei Peng, Hong Shi, Kun Zhou, Tian-xiang Li. Anti-inflammatory effects of Rhodiola rosea L.: A review, Biomedicine & Pharmacotherapy. Volume 121, 2020, 109552. ISSN 0753-3322. https://doi.org/10.1016/j.biopha.2019.109552.
126. Amsterdam J D, Panossian A G. Rhodiola rosea L. as a putative botanical antidepressant. Phytomedicine. 2016 Jun. 15; 23 (7): 770-83. doi: 10.1016/j.phymed.2016.02.009. Epub 2016 Feb. 24. PMID: 27013349.
127. Božović M, Ragno R. Calamintha nepeta (L.) Savi and its Main Essential Oil Constituent Pulegone: Biological Activities and Chemistry. Molecules. 2017; 22 (2): 290. Published 2017 Feb. 14. https://doi.org/10.3390/molecules22020290
128. Pharmacological Activity of (R)-(+)-Pulegone, a Chemical Constituent of Essential Oils, Damião P. de Sousaa, et al. 66 c, 353-359 (2011); received Apr. 24, 2010/Mar. 10, 2011
129. Esatbeyoglu T, Ewald P, Yasui Y, et al. Chemical Characterization, Free Radical Scavenging, and Cellular Antioxidant and Anti-Inflammatory Properties of a Stilbenoid-Rich Root Extract of Vitis vinifera. Oxid Med Cell Longev. 2016; 2016:8591286. https://doi.org/10.1155/2016/8591286
130. Hassan M A, Xu T, Tian Y, Zhong Y, Ali F A Z, Yang X, Lu B. Health benefits and phenolic compounds of Moringa oleifera leaves: A comprehensive review. Phytomedicine. 2021 December; 93:153771. https://doi.org/10.1016/j.phymed.2021.153771. Epub 2021 Oct. 1.
131. Oyeleye S I, Ojo O R, Oboh G. Moringa oleifera leaf and seed inclusive diets influenced the restoration of biochemicals associated with erectile dysfunction in the penile tissue of STZ-induced diabetic male rats treated with/without Acarbose drug. J Food Biochem. 2021 March; 45 (3): e13323. https://doi.org/10.1111/jfbc.13323. Epub 2020 Jun. 15.
132. John L. Wallace, PROSTAGLANDINS, NSAIDS, AND CYTOPROTECTION, Gastroenterology Clinics of North America, Volume 21, Issue 3, 1992, Pages 631-641, ISSN 0889-8553, https://doi.org/10.1016/S0889-8553 (21) 00052-2.
133. Structural requirements for time-dependent inhibition of prostaglandin biosynthesis by anti-inflammatory drugs. L H Rome and W E LandsAuthors Info & Affiliations, Dec. 1, 1975, 72 (12) 4863-4865, https://doi.org/10.1073/pnas.72.12.4863
134. Dull, A.-M.; Moga, M. A.; Dimienescu, O. G.; Sechel, G.; Burtea, V.; Anastasiu, C. V. Therapeutic Approaches of Resveratrol on Endometriosis via Anti-Inflammatory and Anti-Angiogenic Pathways. Molecules 2019, 24, 667. https://doi.org/10.3390/molecules24040667
135. Stochino Loi E, Pontis A, Cofelice V, Pirarba S, Fais M F, Daniilidis A, Melis I, Paoletti A M, Angioni S. Effect of ultramicronized-palmitoylethanolamide and co-micronized palmitoylethanolamide/polydatin on chronic pelvic pain and quality of life in endometriosis patients: An open-label pilot study. Int J Womens Health. 2019 Aug. 12; 11:443-449. doi: 10.2147/IJWH.S204275. PMID: 31496832; PMCID: PMC6697671.

1. A composition comprising at least one N-acylethanolamine (NAE) and at least one of a cannabinoid,
wherein the at least one N-acylethanolamine (NAE) is N-Palmitoylethanolamide (PEA) or an analog or derivative thereof;
wherein the at least one cannabinoid is one or more of Cannabidiol (CBD), Cannabigerol (CBG), Cannabidiolic Acid (CBDA) and Tetrahydrocannabinol (THC);
and wherein the composition if formulated for use for treatment or prevention of pain and/or pain-related symptoms in a subject.

2-6. (canceled)

7. The composition of claim 1, wherein the at least one of a cannabinoid comprises 1, 2, 3, 4 or more cannabinoids.

8-16. (canceled)

17. The composition of claim 1, wherein the at least one N-acylethanolamine (NAE) and the at least one of a cannabinoid are formulated as a pharmaceutical composition suitable for administration to a human subject.

18. The composition of claim 17, comprising one or more pharmaceutically acceptable carriers, excipients or diluents.

19. The composition of claim 17, wherein the composition is formulated for administration by oral, intranasal, inhalation, intrabronchial, parenteral, dermal, transdermal, intravenous, intramuscular, subcutaneous, intraperitoneal, vaginal, rectal, sublingual, buccal, intracranial, epidural, or intratracheal delivery.

20. The composition of claim 17, wherein the composition is formulated for administration in a dosage form comprising a film, a tablet, a liquid dosage form, a hard gelatin capsule, a soft gelatin capsule, gel capsules, a non-gelatin capsule, an HPMC capsule, an inhalant, an injectable, medical patches, topicals, creams, varnishes, sublingual oils, sprays, edibles, a transdermal, a buccal, microneedles, a sublingual patch, a rectal or a vaginal suppository, tampons, cigarettes, vaporizer liquids, nasal preparations, preparations containing micro and/or nano-emulsions, or preparations containing micro and/or nano-particles.

21. The composition of claim 17, wherein the composition is formulated for administration in a dosage form suitable for sustained release, or extended release, or a combined sustained release and extended release dosage forms, or in an immediate release dosage forms, or a combined sustained release and immediate release dosage forms.

22. The composition of claim 17, wherein the composition is formulated as single layer gel-forming film comprising poloxamer 188, glycerol, carboxymethylcellulose (CMC), polyvinyl alcohol (PVA) and active pharmaceutical ingredients comprising the at least one N-acylethanolamine (NAE) and the at least one of a cannabinoid.

23. The composition of claim 22, wherein the gel-forming film comprises (W/W) approximately 5% poloxamer 188, approximately 10% glycerol, approximately 35% CMC, approximately 35% PVA and approximately 15% active pharmaceutical ingredients.

24. The composition of claim 17, wherein the composition is formulated as single or multi-layer gel forming tablet comprising (W/W) approximately 20-45% polyacrylic acid (carbomer), approximately 20-45% hydroxypropyl methylcellulose (HPMC) and approximately 10-60% active pharmaceutical ingredients comprising the at least one N-acylethanolamine (NAE) and the at least one of a cannabinoid.

25. The composition of claim 17, wherein the composition is formulated as a vaginal or rectal suppository that releases active pharmaceutical ingredients comprising the at least one N-acylethanolamine (NAE) and the at least one of a cannabinoid by melting at body temperature or by dissolving in aqueous body fluids.

26. The composition of claim 25, wherein the suppository comprises (W/W) approximately 5-30% active pharmaceutical ingredients and approximately 70-95% suppository bases.

27. The composition of claim 26 wherein the suppository bases comprise one or more of PCCA MBK™ (Fatty Acid), Cocoa Butter NF, PCCA Base A (Polyethylene Glycol 1450 MW, NF), Base F (Witepsol H15™) NF, or bases that are combinations of Polyethylene Glycol NF, polypeg suppository base (Medisca).

28. (canceled)

29. A method for treating or preventing pain in a patient, comprising administering to the patient at least one N-acylethanolamine (NAE) in combination with at least one cannabinoid, wherein the at least one cannabinoid is administered separately, sequentially or simultaneously to the at least one N-acylethanolamine (NAE)
wherein the at least one N-acylethanolamine (NAE) is N-Palmitoylethanolamide (PEA) or an analog or derivative thereof; and
wherein the at least one cannabinoid is one or more of Cannabidiol (CBD), Cannabigerol (CBG), Cannabidiolic Acid (CBDA) and Tetrahydrocannabinol (THC).

30. The method of claim 29, wherein the pain is associated with one or more of primary or secondary dysmenorrhea, endometriosis, pelvic pain, postpartum pain, labor contractions, Polycystic Ovary Syndrome (POCS), oligomenorrhea, hypermenorrhea, metrorrhagia, interstitial cystitis and urinary tract infection pain, pain associated with sexual intercourse, abdominal pain, sexual pain disorder, dyspareunia, muscle pain, migraine, headache, cluster headache, muscle spasm, cramps, hemorrhoids, bone pain, joint pain, arthritis, vulvar pain, haemorrhoidal pain, chronic pain disorder (nociceptive pain, neuropathic pain, chronic back or leg pain.

31. A method for preventing or treating pain-related symptoms in a patient, comprising administering to the patient at least one N-acylethanolamine (NAE) in combination with at least one cannabinoid, wherein the at least one of a cannabinoid is administered separately, sequentially or simultaneously to the at least one N-acylethanolamine (NAE)
wherein the at least one N-acylethanolamine (NAE) is N-Palmitoylethanolamide (PEA) or an analog or derivative thereof; and
wherein the at least one cannabinoid is one or more of Cannabidiol (CBD), Cannabigerol (CBG), Cannabidiolic Acid (CBDA) and Tetrahydrocannabinol (THC).

32. The method of claim 31, wherein the pain-related symptoms comprise one or more of nausea, fatigue, bowel symptoms, hot flashes, facial flushing, night sweat, osteoporosis, indigestion, digestive problems, intestinal disorders, constipation, diarrhea, bladder symptoms, and one or more symptoms caused by pelvic muscle pain or spasm, chronic pelvic pain, pain related to sexual intercourse or pain associated with gynecological cancers.

33. The method of claim 31, wherein the treatment of pain and/or pain-related symptoms comprises alleviating the pain or pain-related symptoms.

34. The method according to any one of claim 31, wherein the treatment of the pain and/or pain-related symptoms comprises managing the pain or pain-related symptoms.

35. The method of claim 31, wherein the treatment of pain and/or pain-related symptoms comprises reducing the pain and/or pain-related symptoms from a more severe state to a less severe state.

36-49. (canceled)

50. A method of treating primary or secondary dysmenorrhea or endometriosis-related pain in a human subject comprising administering a composition as defined in claim 17 to a vaginal mucosal membrane of the subject.

51. A method of treating hemorrhoidal pain in a human subject comprising administering a composition as defined in claim 17 to a rectal mucosal membrane of the subject.

52. A method of treating vulvodynia or vulvar pain in a human subject comprising administering a composition as defined in claim 17 to the vulva of the subject.

53-103. (canceled)

104. The composition according to claim 1, wherein the composition exhibits synergistic inhibition of Cox-1 or Cox-2 enzymes.

105. The composition according to claim 1, wherein the composition exhibits synergistic inhibition of production of prostaglandins.

106-107. (canceled)

108. A method of inhibiting production of prostaglandins in a subject, comprising administering to the subject at least one N-acylethanolamine (NAE) in combination with at least one cannabinoid, wherein the at least one cannabinoid is administered separately, sequentially or simultaneously to the at least one N-acylethanolamine
wherein the at least one N-acylethanolamine (NAE) is N-Palmitoylethanolamide (PEA) or an analog or derivative thereof;
wherein the at least one cannabinoid is one or more of Cannabidiol (CBD), Cannabigerol (CBG), Cannabidiolic Acid (CBDA) and Tetrahydrocannabinol (THC);
and wherein the composition if formulated for use for treatment or prevention of pain and/or pain-related symptoms in a subject.

109-115. (canceled)

116. The composition of claim 1, wherein the PEA is present in an amount of about 100-800 mg and the at least one cannabinoid is present in an amount of about 5-200 mg.

117. The composition of claim 1, comprising one or more pharmaceutically acceptable carriers to enhance mucosal absorption or bioavailability.

118. The composition of claim 1, wherein the composition is formulated for administration in a dosage form comprising a film, a tablet, a liquid dosage form, a hard gelatin capsule, a soft gelatin capsule, gel capsules, a non-gelatin capsule, an HPMC capsule, an inhalant, an injectable, medical patches, topicals, creams, varnishes, sublingual oils, sprays, edibles, a transdermal, a buccal, microneedles, a sublingual patch, a rectal or a vaginal suppository, tampons, cigarettes, vaporizer liquids, nasal preparations, preparations containing micro and/or nano-emulsions, or preparations containing micro and/or nano-particles for managing pain associated with primary or secondary dysmenorrhea, endometriosis, pelvic pain, postpartum pain, labor contractions, Polycystic Ovary Syndrome (POCS), oligomenorrhea, hypermenorrhea, metrorrhagia, interstitial cystitis and urinary tract infection pain, pain associated with sexual intercourse, abdominal pain, sexual pain disorder, dyspareunia, muscle pain, migraine, headache, cluster headache, muscle spasm, cramps, hemorrhoids, bone pain, joint pain, arthritis, vulvar pain, hemorrhoidal pain, chronic pain disorder (nociceptive pain, neuropathic pain, chronic back or leg pain.

119. The composition as defined in claim 21, wherein the sustained release is over a period of 6-12 hours.

120. The method of claim 29, wherein the pain is associated with one or more of ovarian cancer, cervical cancer, endometrial cancer, vulvar cancer and prostate cancer.
